(19) United States
(12) Patent Application Publication
(10) Pub. No.: US 2025/0302958 A1
(43) Pub. Date: Oct. 2, 2025

(54) IMPROVED CELL THERAPIES

(71) Applicant: UNIKUM THERAPEUTICS APS, København (DK)

(72) Inventors: Rasmus Otkjær BAK, København (DK); Martin Roelsgaard JAKOBSEN, København (DK); Ulrik NIELSEN, København (DK); Mette Louise TREMPENAU, København (DK)

(21) Appl. No.: 18/864,430

(22) PCT Filed: May 10, 2023
(86) PCT No.: PCT/EP2023/062413

§ 371 Date: Nov. 8, 2024

(30) Foreign Application Priority Data
May 10, 2022 (GB) 2206816.7

(51) Int. Cl. A61K 40/19; A61K 40/24; A61K 40/31; A61K 40/42; A61P 35/00; C07K 14/705; C07K 14/725

(52) U.S. Cl. CPC A61K 40/19; A61K 40/24; A61K 40/31; A61K 40/4211; A61P 35/00; C07K 14/70503; C07K 14/7051

(57) ABSTRACT

The present invention relates to methods for treating disease, in particular cancer and autoimmune, inflammatory and infectious diseases, using engineered plasmacytoid dendritic cells and adoptive cell transfer immunotherapies.

### FIELD OF THE INVENTION

The present invention relates to methods for treating disease, in particular cancer and autoimmune, inflammatory and infectious diseases, using engineered cells.

### BACKGROUND OF THE INVENTION

Adoptive cell transfer (ACT) immunotherapy is an established and successful approach to treating cancer, autoimmune, inflammatory and infectious diseases. ACT is the passive transfer of ex vivo grown cells, most commonly immune-derived cells, into a host with the goal of transferring the immunologic functionality and characteristics of the transplant.

One example of ACT is CAR-T therapy. In CAR-T therapy, a T-cell population is obtained from a patient or donor and is engineered to express a chimeric antigen receptor (CAR). The extracellular domain of a typical CAR consists of the VH and VL domains-single-chain fragment variable (scFv)-from the antigen binding sites of a monoclonal antibody that recognises a tumour associated antigen. The scFv is linked to a flexible transmembrane domain followed by an intracellular signalling domain with, for example, a tyrosine-based activation motif such as that from CD3z, and optionally additional activation domains from co-stimulatory molecules such as CD28 and CD137 (41BB), which serve to activate the T-cells and enhance their survival and proliferation. CAR T-cells are administered to the patient and the CAR recognises and binds tumour cells. Binding of cancer cells leads to activation of cytolytic mechanisms in the T-cells, which specifically kill the bound cancer cells. Various preclinical and early-phase clinical trials highlight the efficacy of CAR T cells to treat cancer patients with solid tumours and hematopoietic malignancies.

Other cells have also been proposed for use with CARs. Natural killer cells are a type of cytotoxic lymphocyte that naturally attack virus-infected cells and tumour cells and can be engineered with CARs to form CAR-NK cells. Other phagocytic cells capable of cellular cytotoxicity are also expected to be useful in this context, in particular macrophages.

Obstacles to successful CAR-T and CAR-NK therapy include loss of the targeted antigen from tumours, immunosuppressive cells such as Tregs and myeloid-derived suppressor cells (MDSCs) in the tumour microenvironment, factors in the tumour microenvironment such as adenosine, extracellular potassium, and reactive oxygen species (ROS) that can inhibit the cytolytic activity of T-cells, and tumours that express chemokines for which CAR-T cells do not have receptors.

Plasmacytoid dendritic cells (pDCs) are a rare type of immune cell that are considered to be key in linking the innate and adaptive immune systems. They are known to secrete large quantities of type 1 interferon (IFNs) in response to a viral infection and are key effectors in cellular immunity with the ability to not only initiate immune responses but also to induce tolerance to exogenous and endogenous antigens. WO2018/206577 provides methods for generating populations of pDCs.

There remains a need in the art for improved methods for treating cancer and other diseases.

### SUMMARY OF THE INVENTION

The inventors have developed a method of treatment that utilises the unique properties of plasmacytoid dendritic cells (pDCs) to improve the effectiveness of an ACT immunotherapy. Specifically, the inventors have demonstrated that co-culturing an ACT immunotherapy with pDCs expressing at least one antigen that is bound by a receptor expressed by the ACT immunotherapy improves the ability of the ACT immunotherapy to kill target cells, increases the levels of cytokines produced by the ACT immunotherapy, and improves the proliferation and decreases exhaustion of the ACT immunotherapy. According to the invention, pDCs are engineered to express on their surface an antigen to which a CAR or other receptor expressed by an ACT immunotherapy can bind. The extracellular antigen recognition domain allows the pDCs to specifically target the adoptive cells expressing the CAR or other receptor and improve the immune response of the cells and their ability to survive in the disease microenvironment. The multifaceted nature of pDCs means that they can be used to activate extensive effects on immune responses, for example by secreting pro-inflammatory cytokines, or recruiting and activating immune cells such as cytotoxic lymphocytes. As a consequence, pDCs are expected to exert powerful and pervasive effects, improving the efficacy of an ACT immunotherapy even in disease microenvironments that are refractive to the desired immune response. For example, pDCs express ligands (e.g. CD80 and CD86) that activate co-stimulatory receptors on T cells (e.g. CD28). Accordingly, the inventors have also developed methods utilising pDCs that express at least one receptor that binds an antigen expressed by the ACT immunotherapy, such as a chimeric auto-antibody receptor. The pDCs can be used to enhance the activity of such ACT immunotherapies against auto-reactive immune cells, for example.

In certain embodiments, the pDCs are administered in combination with an ACT immunotherapy. In certain embodiments, the pDCs are administered simultaneously with the ACT immunotherapy. In certain embodiments, the pDCs are administered separately from the ACT immunotherapy. In some embodiments, the pDCs are administered prior to the ACT immunotherapy. In some embodiments, the pDCs are administered subsequent to the ACT immunotherapy. In some embodiments, the pDCs are administered repeatedly in two or more doses subsequent to the ACT immunotherapy.

The Examples demonstrate that pDCs can express antigens. The pDCs maintain their functionality and type I IFN response and can be successfully recognised by target cells, bind and subsequently enhance the cytokine production and proliferation and reduce exhaustion of the target cells, and improve their ability to kill target cells.

In some embodiments, the pDCs are immunogenic. In some embodiments, the pDCs stimulate an immune response against the antigen.

Accordingly, the invention provides a method for treating a disease in a subject, comprising administering plasmacytoid dendritic cells (pDCs) in combination with an ACT immunotherapy, wherein the pDCs express at least one antigen that is bound by a receptor expressed by the ACT immunotherapy. The invention also provides a method for treating a disease in a subject, comprising administering plasmacytoid dendritic cells (pDCs) in combination with an ACT immunotherapy, wherein the pDCs express at least one receptor that binds an antigen expressed by the ACT immunotherapy.

The invention also provides method for treating a disease comprising administering an ACT immunotherapy, wherein the method comprises a step of pre-conditioning the ACT immunotherapy with pDCs expressing at least one antigen that is bound by a receptor expressed by the ACT immunotherapy or with pDCs expressing at least one receptor that binds an antigen expressed by the ACT immunotherapy.

The invention also provides a method of pre-conditioning an ACT immunotherapy comprising co-culturing the ACT immunotherapy with pDCs expressing at least one antigen that is bound by a receptor expressed by the ACT immunotherapy or with pDCs expressing at least one receptor that binds an antigen expressed by the ACT immunotherapy.

In a preferred embodiment, the invention provides a method for treating cancer comprising administering pDCs in combination with an ACT immunotherapy, wherein the pDCs express at least one antigen that is bound by a receptor expressed by the ACT immunotherapy.

In a preferred embodiment, the invention provides a method for treating cancer comprising administering pDCs in combination with an ACT immunotherapy, wherein the ACT immunotherapy comprises CAR T-cells, and wherein the pDCs express at least one antigen that is bound by the CAR expressed by the CAR T-cells.

In certain embodiments, the invention provides a method for treating a CD19+ cancer comprising administering pDCs in combination with an ACT immunotherapy, wherein the ACT immunotherapy comprises anti-CD19 CAR T-cells, and wherein the pDCs express CD19.

In certain embodiments, the invention provides a method for treating a HER2+ cancer comprising administering pDCs in combination with an ACT immunotherapy, wherein the ACT immunotherapy comprises anti-HER2 CAR T-cells, and wherein the pDCs express HER2.

In certain embodiments, the invention provides a method for treating a CD70+ cancer comprising administering pDCs in combination with an ACT immunotherapy, wherein the ACT immunotherapy comprises anti-CD70 CAR T-cells, and wherein the pDCs express CD70.

In certain embodiments, the invention provides a method for treating a CD19+ cancer comprising administering pDCs in combination with an ACT immunotherapy, wherein the ACT immunotherapy comprises anti-CD19 CAR T-cells, and wherein the pDCs express CD19, wherein the CD19 is truncated to disrupt its signalling activity.

In certain embodiments, the invention provides a method for treating a HER2+ cancer comprising administering pDCs in combination with an ACT immunotherapy, wherein the ACT immunotherapy comprises anti-HER2 CAR T-cells, and wherein the pDCs express HER2, wherein the HER2 is truncated to disrupt its signalling activity.

In certain embodiments, the invention provides a method for treating a CD70+ cancer comprising administering pDCs in combination with an ACT immunotherapy, wherein the ACT immunotherapy comprises anti-CD70 CAR T-cells, and wherein the pDCs express CD70, wherein the CD70 is truncated to disrupt its signalling activity.

In a preferred embodiment, the invention provides a method for treating cancer comprising administering pDCs in combination with an ACT immunotherapy, wherein the ACT immunotherapy comprises CAR T-cells, and wherein the pDCs an antigen truncated to disrupt its signalling activity.

In a preferred embodiment, the invention provides a method for treating cancer comprising administering an ACT immunotherapy, wherein the method comprises a step of pre-conditioning the ACT immunotherapy with pDCs expressing at least one antigen that is bound by a receptor expressed by the ACT immunotherapy.

In a preferred embodiment, the invention provides a method of treating cancer comprising administering an ACT immunotherapy, wherein the ACT immunotherapy comprises CAR T-cells, and wherein the method comprises a step of pre-conditioning the ACT immunotherapy with pDCs expressing at least one antigen that is bound by the CAR expressed by the CAR T-cells.

In certain embodiments, the invention provides a method of treating a CD19+ cancer comprising administering an ACT immunotherapy, wherein the ACT immunotherapy comprises anti-CD19 CAR T-cells, and wherein the method comprises a step of pre-conditioning the ACT immunotherapy with pDCs expressing CD19.

In certain embodiments, the invention provides a method of treating a CD19+ cancer comprising administering an ACT immunotherapy, wherein the ACT immunotherapy comprises anti-CD19 CAR T-cells, and wherein the method comprises a step of pre-conditioning the ACT immunotherapy with pDCs expressing CD19, wherein the CD19 is truncated to disrupt its signalling activity.

In certain embodiments, the invention provides a method of treating a HER2+ cancer comprising administering an ACT immunotherapy, wherein the ACT immunotherapy comprises anti-HER2 CAR T-cells, and wherein the method comprises a step of pre-conditioning the ACT immunotherapy with pDCs expressing HER2.

In certain embodiments, the invention provides a method of treating a HER2+ cancer comprising administering an ACT immunotherapy, wherein the ACT immunotherapy comprises anti-HER2 CAR T-cells, and wherein the method comprises a step of pre-conditioning the ACT immunotherapy with pDCs expressing HER2, wherein the HER2 is truncated to disrupt its signalling activity.

In certain embodiments, the invention provides a method of treating a CD70+ cancer comprising administering an ACT immunotherapy, wherein the ACT immunotherapy comprises anti-CD70 CAR T-cells, and wherein the method comprises a step of pre-conditioning the ACT immunotherapy with pDCs expressing CD70.

In certain embodiments, the invention provides a method of treating a CD70+ cancer comprising administering an ACT immunotherapy, wherein the ACT immunotherapy comprises anti-CD70 CAR T-cells, and wherein the method comprises a step of pre-conditioning the ACT immunotherapy with pDCs expressing CD70, wherein the CD70 is truncated to disrupt its signalling activity.

In preferred embodiments of any method of the invention for treating cancer, the treatment reduces tumour volume. In preferred embodiments of any method of the invention for treating cancer, the treatment reduces tumour weight. In a preferred embodiment, the invention provides a method of treating cancer comprising administering pDCs in combination with an ACT immunotherapy, wherein treatment reduces tumour volume. In a preferred embodiment, the invention provides a method of treating cancer comprising administering pDCs in combination with an ACT immunotherapy, wherein treatment reduces tumour weight.

In a preferred embodiment, the invention provides a method of treating a disease in a subject comprising administering an ACT immunotherapy to a patient that previously received and/or is scheduled to receive administration of pDCs, wherein the pDCs express at least one antigen that is bound by a receptor expressed by the ACT immunotherapy, or wherein the pDCs express at least one receptor that binds an antigen expressed by the ACT immunotherapy.

In a preferred embodiment, the invention provides a method of treating a disease in a subject comprising administering pDCs to a patient that previously received and/or is scheduled to receive administration of an ACT immunotherapy, wherein the pDCs express at least one antigen that is bound by a receptor expressed by the ACT immunotherapy, or wherein the pDCs express at least one receptor that binds an antigen expressed by the ACT immunotherapy.

In a preferred embodiment, the invention provides a method for increasing the activity of an ACT immunotherapy in the treatment of a disease, comprising administering pDCs, wherein the pDCs express at least one antigen that is bound by a receptor expressed by the ACT immunotherapy, or wherein the pDCs express at least one receptor that binds an antigen expressed by the ACT immunotherapy.

In a preferred embodiment, the invention provides a method for increasing the proliferation of an ACT immunotherapy in the treatment of a disease, comprising administering pDCs, wherein the pDCs express at least one antigen that is bound by a receptor expressed by the ACT immunotherapy, or wherein the pDCs express at least one receptor that binds an antigen expressed by the ACT immunotherapy.

In a preferred embodiment, the invention provides pDCs for use in increasing the activity of an ACT immunotherapy in the treatment of a disease, wherein the pDCs are administered in combination with the ACT immunotherapy and wherein the pDCs express at least one antigen that is bound by a receptor expressed by the ACT immunotherapy, or wherein the pDCs express at least one receptor that binds an antigen expressed by the ACT immunotherapy.

In a preferred embodiment, the invention provides pDCs for use in increasing the proliferation of an ACT immunotherapy in the treatment of a disease, wherein the pDCs are administered in combination with the ACT immunotherapy and wherein the pDCs express at least one antigen that is bound by a receptor expressed by the ACT immunotherapy, or wherein the pDCs express at least one receptor that binds an antigen expressed by the ACT immunotherapy.

Further embodiments of the invention are provided in the numbered paragraphs below:
1 A method for treating a disease in a subject, comprising administering plasmacytoid dendritic cells (pDCs) in combination with an adoptive cell transfer immunotherapy, wherein the pDCs express at least one antigen that is bound by a receptor expressed by the adoptive cell transfer immunotherapy, or wherein the pDCs express at least one receptor that binds an antigen expressed by the adoptive cell transfer immunotherapy.
2. The method of embodiment 1, wherein the pDCs are administered simultaneously with the adoptive cell transfer immunotherapy.
3 The method of embodiment 1, wherein the pDCs are administered separately from the adoptive cell transfer immunotherapy, such as wherein the pDCs are administered before the adoptive cell transfer immunotherapy, or wherein the adoptive cell transfer immunotherapy is administered before the pDCs.
4. A method for treating a disease in a subject comprising administering an adoptive cell transfer immunotherapy, wherein the method comprises a step of pre-conditioning the adoptive cell transfer immunotherapy before administration, and wherein the step of pre-conditioning comprises co-culturing the adoptive cell transfer immunotherapy with pDCs expressing at least one antigen that is bound by a receptor expressed by the adoptive cell transfer immunotherapy, or with pDCs expressing at least one receptor that binds an antigen expressed by the adoptive cell transfer immunotherapy.
5. A method of pre-conditioning an adoptive cell transfer immunotherapy comprising co-culturing the adoptive cell transfer immunotherapy with pDCs expressing at least one antigen that is bound by a receptor expressed by the adoptive cell transfer immunotherapy, or with pDCs expressing at least one receptor that binds an antigen expressed by the adoptive cell transfer immunotherapy.
6. The method of embodiment 4 or embodiment 5, wherein the ratio of adoptive cell transfer immunotherapy cells to pDCs in the co-culturing is between 2:1 and 1:2, such as between 1.5:1 and 1:1.5, or between 1.2:1 and 1:1.2, or preferably about 1:1.
7. The method of embodiment 4 or embodiment 5, wherein the ratio of adoptive cell transfer immunotherapy cells to pDCs in the co-culturing is between 1:0.4 and 1:01, such as between 1:0.3 and 1:0.1, or preferably about 1:0.2.
8. The method of any one of embodiments 4-7, wherein the step of pre-conditioning increases the levels of cytokines produced by the adoptive cell transfer immunotherapy.
9. The method of embodiment 8, wherein the cytokines comprise IL-2, TFNα, and/or IFNγ.
10. The method of embodiment 8 or embodiment 9, wherein the step of pre-conditioning increases the levels of cytokines produced by the adoptive cell transfer immunotherapy when compared to a corresponding method comprising a step of co-culturing the adoptive cell transfer immunotherapy with pDCs not expressing at least one antigen that is bound by a receptor expressed by the adoptive cell transfer immunotherapy.
11. The method of embodiment 8 or 9, wherein the step of pre-conditioning increases the levels of cytokines produced by the adoptive cell transfer immunotherapy when compared to a corresponding method comprising a step of co-culturing the adoptive cell transfer immunotherapy with tumour cells expressing at least one antigen that is bound by a receptor expressed by the adoptive cell transfer immunotherapy.
12. The method of any one of embodiments 4-11, wherein the step of pre-conditioning increases the proliferation of the adoptive cell transfer immunotherapy.
13. The method of embodiment 12, wherein the step of pre-conditioning increases the proliferation of the adoptive cell transfer immunotherapy when compared to a corresponding method comprising the step of co-culturing the adoptive cell transfer immunotherapy with pDCs not expressing at least one antigen that is bound by a receptor expressed by the adoptive cell transfer immunotherapy.
14. The method of embodiment 12, wherein the step of pre-conditioning increases the proliferation of the adoptive cell transfer immunotherapy when compared to a corresponding method comprising the step of co-culturing the adoptive cell transfer immunotherapy with tumour cells expressing at least one antigen that is bound by a receptor expressed by the adoptive cell transfer immunotherapy.
15. The method of any one of embodiments 4-14, wherein the step of pre-conditioning decreases the level of exhaustion, such as measured by expression of exhaustion markers, of the adoptive cell transfer immunotherapy.
16. The method of embodiment 15, wherein the step of pre-conditioning decreases the level of exhaustion, such as measured by expression of exhaustion markers, of the adoptive cell transfer immunotherapy when compared to a corresponding method comprising the step of co-culturing the adoptive cell transfer immunotherapy with pDCs not expressing at least one antigen that is bound by a receptor expressed by the adoptive cell transfer immunotherapy.
17. The method of embodiment 15, wherein the step of pre-conditioning decreases the level of exhaustion, such as measured by expression of exhaustion markers, of the adoptive cell transfer immunotherapy when compared to a corresponding method comprising the step of co-culturing the adoptive cell transfer immunotherapy with tumour cells expressing at least one antigen that is bound by a receptor expressed by the adoptive cell transfer immunotherapy.
18. The method of any one of embodiments 4-17, further comprising a step of purifying the adoptive cell transfer immunotherapy.
19. The method of any one of embodiments 4-18, further comprising a step of formulating the adoptive cell transfer immunotherapy with a pharmaceutically acceptable excipient.
20. The method of any one of the preceding embodiments, wherein the disease is:
   (a) a cancer;
   (b) an autoimmune disease;
   (c) an inflammatory disease; or
   (d) an infectious disease.
21. The method of embodiment 20a, wherein the cancer is a solid cancer, such as a cancer selected from the group consisting of: bone cancer, breast cancer, pancreatic cancer, skin cancer, cancer of the head or neck, cutaneous or intraocular malignant melanoma, uterine cancer, ovarian cancer, prostate cancer, rectal cancer, cancer of the anal region, colon cancer, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, glioblastoma, pituitary adenoma, Kaposi's sarcoma, epidermoid cancer and squamous cell cancer.
22. The method of embodiment 20a, wherein the cancer is selected from the group consisting of acute lymphoblastic leukemia (ALL) (including non T cell ALL), acute myeloid leukemia, B cell prolymphocytic leukemia, B-cell acute lymphoid leukemia (“BALL”), blastic plasmacytoid dendritic cell neoplasm, Burkitt s lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell-or a large cell-follicular lymphoma, splenic marginal zone lymphoma (SMZL), systemic amyloid light chain amyloidosis, T-cell acute lymphoid leukemia (“TALL”), T-cell lymphoma, transformed follicular lymphoma, and Waldenstrom macroglobulinemia.
23. The method of embodiment 20a or embodiment 22 or embodiment 23, wherein the pDCs express at least one antigen that is bound by a receptor expressed by the adoptive cell transfer immunotherapy and the at least one antigen is a tumour-associated antigen expressed by the cancer to be treated.
24 The method of embodiment 23, wherein the at least one antigen is selected from:
   (a) the group consisting of CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPA7, PSCA, EGFR, EGFRVIII, ROR1, mesothelin, CD33/IL3Ra, c-Met, CD37, PSMA, Glycolipid F77, GD-2, gp100, NY-ESO-1 TCR, FRalpha, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or a combination thereof; or
   (b) the group consisting of 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD70, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumour antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1, HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen, CD38, insulin growth factor (IGF1)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumour-specific peptide epitope, M-CSF, melanoma-associated antigen, mesothelin, MN-CA IX, MUC-1, mut hsp70-2, mutated p53, mutated p53, mutated ras, neutrophil elastase, NKG2D, Nkp30, NY-ESO-1, p53, PAP, prostase, prostate specific antigen (PSA), prostate-carcinoma tumour antigen-1 (PCTA-1), prostate-specific antigen protein, STEAP1, STEAP2, PSMA, RAGE-1, ROR1, RUI, RU2 (AS), surface adhesion molecule, survivin, telomerase, TAG-72, the extra domain A (EDA) and extra domain B (EDB) of fibronectin and the Al domain of tenascin-C (TnC Al), thyroglobulin, tumour stromal antigens, vascular endothelial growth factor receptor-2 (VEGFR2), virus-specific surface antigen such as an HIV-specific antigen, or a combination thereof.
25. The method of embodiment 20b, wherein the autoimmune disease is selected from the group consisting of type 1 diabetes, thyroid autoimmune disease, Addison's adrenal insufficiency, oophoritis, orchitis, lymphocytic hypophysitis, autoimmune hypoparathyroidism, autoimmune hypoparathyroidism, Goodpasture's disease, autoimmune myocarditis, membranous nephropathy, autoimmune hepatitis, ulcerative colitis, Crohn's disease, multiple sclerosis, myasthenia gravis, neuromyelitis optica, encephalitis and Sjögren's syndrome.
26. The method of embodiment 20c, wherein the inflammatory disease is selected from the group consisting of cystic fibrosis, chronic inflammatory intestinal diseases like, for example, ulcerative colitis or Crohn's disease, vasculitis, in particular Kawasaki disease, chronic bronchitis, inflammatory arthritis diseases like, for example, psoriatic arthritis, osteoarthritis, rheumatoid arthritis, and systemic onset juvenile rheumatoid arthritis (SOJRA, Still's disease), graft-versus-host disease, asthma, psoriasis, systemic lupus erythematosus, obesity and inflammatory vascular disease and allograft rejection.
27. The method of embodiment 20b, 20c, 25 or 26, wherein the pDCs express at least one receptor that binds an antigen expressed by the adoptive cell transfer immunotherapy and the antigen is recognised by auto-reactive antibodies that cause the autoimmune or inflammatory disease.
28 The method of embodiment 20b, 20c, 25 or 26, wherein the pDCs express at least one antigen that is bound by a receptor expressed by the adoptive cell transfer immunotherapy and the antigen is expressed on the surface of immune cells that cause the autoimmune or inflammatory disease.
29. The method of embodiment 20b or embodiment 20c, wherein the disease is transplant rejection or graft-versus-host disease (GVHD).
30 The method of embodiment 29, wherein the pDCs express at least one receptor that binds an antigen expressed by the adoptive cell transfer immunotherapy and the antigen is recognised by auto-reactive antibodies that cause the transplant rejection or GVHD.
31. The method of embodiment 29, wherein the pDCs express at least one antigen that is bound by a receptor expressed by the adoptive cell transfer immunotherapy and the antigen is expressed on the surface of B-cells that cause the transplant rejection or GVHD.
32. The method of embodiment 20 (d), wherein the infectious disease is a viral or fungal infection, such as infection of Influenza, Coronavirus, RSV, Measles, Parainfluenza, Zikavirus, Dengue virus, HIV, HBV, HCV, human cytomegalovirus (CMV), Epstein-Barr virus (EBV), Aspergillus spp., such as Aspergillus fumigatus, Candida spp., such as C. albicans, Mucorales, Cryptococcus, spp., such as Cryptococcus neoformans or Cryptococcus gattii, or Pneumocystis jirovecii, optionally wherein the infection is a chronic infection.
33. The method of embodiment 32, wherein the pDCs express at least one antigen that is bound by a receptor expressed by the adoptive cell transfer immunotherapy and the at least one antigen is a pathogen antigen.
34. The method of embodiment 33, wherein the at least one antigen is selected from: Influenza hemagglutinin antigens, Influenza neuraminidase antigens, coronavirus spike protein, RSV F protein, MeV N protein, Parainfluenza hemagglutinin antigens, Parainfluenza neuraminidase antigens, ZIKV E, NS1, NS3, NS4B, and NS5 proteins, Dengue virus C protein, M protein, E protein, and NS1 protein, gp120, gp41, Env, HBV surface antigen, HBV-surface proteins S and L, HCV E2 glycoprotein, CMV glycoprotein B, fungal beta glucan.
35 The method of any of the preceding embodiments, wherein the pDCs express at least one antigen that is bound by a receptor expressed by the adoptive cell transfer immunotherapy and wherein the at least one antigen is modified to disrupt its signalling activity.
36. The method of any one of the preceding embodiments, wherein the pDCs express at least one antigen that is bound by a receptor expressed by the adoptive cell transfer immunotherapy and wherein the at least one antigen is truncated to remove one or more intracellular signalling domains.
37. The method of any one of the preceding embodiments, wherein the pDCs express at least one antigen that is bound by a receptor expressed by the adoptive cell transfer immunotherapy and wherein the at least one antigen is truncated to remove one or more intracellular effector domains.
38. The method of any one of the preceding embodiments, wherein the pDCs comprise a heterologous nucleic acid encoding the at least one antigen or at least one receptor.
39. The method of embodiment 38, wherein the heterologous nucleic acid is selected from the group consisting of a viral construct, a plasmid, a cosmid, an mRNA.
40. The method of embodiment 39, wherein the viral construct is a lentiviral vector.
41. The method of embodiment 39 or embodiment 40, wherein the viral construct comprises an expression cassette between two ITRs.
42. The method of embodiment 38 wherein the heterologous nucleic acid is introduced using a site-specific DNA editing, such as CRISPR/Cas.
43. The method of any of embodiments 38 to 42, wherein the heterologous nucleic acid is an expression cassette comprising one or more transgenes encoding the at least one antigen operably linked to a promoter.
44 The method of any one of the preceding embodiments, wherein the adoptive cell transfer immunotherapy comprises T-cells, natural killer cells, dendritic cells, macrophages or tumour-infiltrating lymphocytes (TILs).
45. The method of any one of the preceding embodiments, wherein the adoptive cell transfer immunotherapy comprises cells expressing a chimeric antigen receptor, a T-cell receptor, a synthetic Notch receptor, or a chimeric auto-antibody receptor.
46. The method of any one of the preceding embodiments, wherein the pDCs have been extracted from blood or bone marrow.
47. The method of any one of the preceding embodiments, wherein the pDCs have been differentiated in vitro from HSPCs, optionally wherein the HSPCs have been obtained from blood or bone marrow, or wherein the HSPCs have been differentiated in vitro from induced pluripotent stem cells (iPSCs) or embryonic stem cells (ESCs).
48. The method of any one of the preceding embodiments, wherein the method further comprises a step of priming the pDCs.
49. The method of embodiment 48, wherein the step of priming the pDCs comprises incubating the pDCs with type I IFN and/or type II IFN.
50. The method of embodiment 49, wherein the pDCs are incubated with the type I IFN and/or type II IFN for 24 hours.
51. The method of any one of the preceding embodiments, further comprising a step of producing the pDCs, wherein the step of producing the pDCs comprises the steps of:
   (a) transfecting or transducing HSPCs with a vector comprising an expression cassette comprising a transgene encoding the at least one antigen operably linked to a promoter; and
   (b) differentiating the HSPCs into pDCs.
52. The method of any one of embodiments 1-50, further comprising a step of producing the pDCs, wherein the step of producing the pDCs comprises the steps of:
   (a) differentiating HSPCs into pDCs; and
   (b) transfecting or transducing the pDCs with a vector comprising an expression cassette comprising a transgene encoding the at least one antigen operably linked to a promoter.
53. The method of embodiment 51 or embodiment 52, wherein the vector is a lentiviral vector.
54. The method of embodiment 51 or embodiment 52, wherein the vector is an mRNA, optionally encapsulated in a lipid nanoparticle.
55. The method of any one of embodiments 51-54, wherein the step of differentiating the HSPCs comprises incubating said HSPCs in one or more media, which media may typically comprise one or more cytokines, growth factors, interferons (IFNs) and/or aryl hydrocarbon receptor (AHR) antagonists (such as stemregenin-1), whereby said HSPCs are differentiated into precursor-pDCs and into pDCs.
56. The method of any one of embodiments 51-55, further comprising a step of purifying the pDCs.
57. The method of any one of embodiments 51-56, further comprising a step of formulating the pDCs with a pharmaceutically acceptable excipient.
58. A method of treating a disease in a subject comprising administering an adoptive cell transfer immunotherapy to a patient that previously received and/or is scheduled to receive administration of pDCs, wherein the pDCs express at least one antigen that is bound by a receptor expressed by the adoptive cell transfer immunotherapy, or wherein the pDCs express at least one receptor that binds an antigen expressed by the adoptive cell transfer immunotherapy.
59. A method of treating a disease in a subject comprising administering pDCs to a patient that previously received and/or is scheduled to receive administration of an adoptive cell transfer immunotherapy, wherein the pDCs express at least one antigen that is bound by a receptor expressed by the adoptive cell transfer immunotherapy, or wherein the pDCs express at least one receptor that binds an antigen expressed by the adoptive cell transfer immunotherapy.
60. A method for increasing the activity of an adoptive cell transfer immunotherapy in the treatment of a disease, comprising administering pDCs, wherein the pDCs express at least one antigen that is bound by a receptor expressed by the adoptive cell transfer immunotherapy, or wherein the pDCs express at least one receptor that binds an antigen expressed by the adoptive cell transfer immunotherapy.
61. A method for increasing the proliferation of an adoptive cell transfer immunotherapy in the treatment of a disease, comprising administering pDCs, wherein the pDCs express at least one antigen that is bound by a receptor expressed by the adoptive cell transfer immunotherapy, or wherein the pDCs express at least one receptor that binds an antigen expressed by the adoptive cell transfer immunotherapy.
62. pDCs for use in increasing the activity of an adoptive cell transfer immunotherapy in the treatment of a disease, wherein the pDCs are administered in combination with the adoptive cell transfer immunotherapy and wherein the pDCs express at least one antigen that is bound by a receptor expressed by the adoptive cell transfer immunotherapy, or wherein the pDCs express at least one receptor that binds an antigen expressed by the adoptive cell transfer immunotherapy.
63. pDCs for use in increasing the proliferation of an adoptive cell transfer immunotherapy in the treatment of a disease, wherein the pDCs are administered in combination with the adoptive cell transfer immunotherapy and wherein the pDCs express at least one antigen that is bound by a receptor expressed by the adoptive cell transfer immunotherapy, or wherein the pDCs express at least one receptor that binds an antigen expressed by the adoptive cell transfer immunotherapy.

### BRIEF DESCRIPTION OF THE FIGURES

FIG. 1—Exemplary process for the generation of U-pDC^{ENHANCE} cells. CD34⁺ hematopoietic stem and progenitor cells (HSPCs) are transduced with a lentiviral vector encoding the truncated CAR-targeting antigen (CAR-antigen). The CAR-antigen-expressing CD34⁺ HSPCs are subsequently differentiated into U-pDCs termed U-pDC^{ENHANCE} cells. The U-pDC^{ENHANCE} cells can be used fresh or cryopreserved for later use.

FIG. 2—Structure of lentiviral vector encoding the truncated antigen. Schematic of a lentiviral construct encoding truncated CAR-antigen (here CD19 is used as an example albeit any relevant antigen could be used). The intracellular domains of the CAR-target protein are truncated to render the protein signalling incompetent to not potentially affect the U-pDC biology. Expression of the extracellular domains on the U-pDC surface allows CAR T cell binding and subsequent activation of the CAR T cell.

FIG. 3—Exemplary effects of U-pDC^{ENHANCE} and CAR T cell interaction in vivo. U-pDC^{ENHANCE} cells migrate to lymphoid organs e.g., lymph nodes, where they are recognized and bound by the corresponding CAR T cells. This binding facilitates the delivery of potent immune activating signals to the CAR T cells through the co-stimulatory molecules naturally expressed on U-pDCs (e.g., CD40, CD80, and CD86). This leads to strong activation of the CAR T cells resulting in increased proliferation, cytotoxic activity, cytokine production, and decreased exhaustion. The cytokine release of e.g., IFNγ, IL-2, and TNFα by the CAR T cells can in turn facilitate activation of endogenous anti-tumour immune cells. Together these effects support a more efficient tumour regression.

FIG. 4—Exemplary process for boosting CAR T cell therapy by U-pDC^{ENHANCE} administration. Fresh or cryo-preserved U-pDC^{ENHANCE} cells (autologous or allogeneic donor) can be administered as a boosting regiment following CAR T cell therapy. Simultaneous administration of U-pDC^{ENHANCE} cells and CAR T cell is also an option. U-pDC^{ENHANCE} cells migrate to lymphoid organs and facilitate a strong activation of the CAR T cells (increased proliferation, cytotoxic activity, and cytokine production) overcoming the immune-suppressing and-exhausting microenvironment associated with solid tumours. This stimulation also reinvigorates exhausted CAR T cells. Additionally, the pro-inflammatory cytokine release from the U-pDC-activated CAR T cells facilitates activation of endogenous anti-tumour immune cells (e.g., NK and T cells). Together these effects support a more efficient tumour regression.

FIG. 5—U-pDCs can be modified to express a CAR-antigen. A) Representative flow cytometry plots showing the expression of truncated CD19 (tCD19) in CD34⁺ HSPC-derived U-pDCs. B) Bar graph showing the percentage of tCD19⁺ U-pDCs generated from six donors (buffy coat or cord-blood derived CD34⁺ HSPCs).

FIG. 6—Expansion and immunophenotype of U-pDCs genetically modified to express truncated CD19. tCD19⁺ lentivirally transduced (tCD19) or untransduced (Mock) CD34⁺ cells were differentiated into U-pDCs (A), and subsequently primed with type I and II IFN for 24 hours and analyzed for pDC-marker expression by flow cytometry (B). A) Growth curve comparing the cell expansion of Mock versus tCD19-transduced conditions during differentiation from CD34⁺ cells to U-pDCs for individual cord blood and buffy coat (BC) donors. B) Bar plot showing the frequency of IFN-primed Mock-and tCD19-U-pDCs with pDC-marker expression (CD123 and CD303).

FIG. 7—Type I IFN responses of U-pDCs genetically modified to express truncated CD19. Primed or non-primed U-pDCs, where 95% of the cells express a truncated CD19 (U-pDC^{ENHANCE}), or U-pDCs not expressing the construct (Mock U-pDC), were stimulated for 20 hours with agonists directed against TLR7 (R837) or remained unstimulated. Supernatants were subsequently harvested, and IFNα-responses were quantified using ELISA. Exemplary data from one donor. Co-cultures were performed in technical duplicates. The data show comparable ability to respond to TLR7 agonist between Mock U-pDCs and U-PDC^{ENHANCE} cells.

FIG. 8—CAR T cell activation after U-pDC^{ENHANCE} co-culture. tCD19⁺ U-pDC^{ENHANCE} and Mock U-pDC cells were thawed and cultured 24 hours prior to IFN-priming. After 24 hours priming, the cells were co-cultured with anti-CD19 CAR T cells (A) or Mock T cells (B) at a T cell: Enhance cell ratio of 1:1 or 1:0.2. CAR-T cells co-cultured with CD19-expressing target tumour cells (NALM6) were included as activation control. Enhance cells and controls were additionally seeded without CAR T cells (0:1) to exclude potential background. After 24 hours, supernatants were harvested and levels of the T cell activation associated cytokines (IL-2, TFNα, and IFNyγ) were quantified using ELISA. Exemplary data from one allogeneic donor experiment. Co-cultures were performed in technical duplicates.

FIG. 9—CAR T cell proliferation after U-pDC^{ENHANCE} co-culture. Anti-CD19-CAR T cells were stained with CellTrace Yellow dye and co-cultured with mock U-pDCs or tCD19-U-pDCs^{ENHANCE} at a T cell: Enhance cell ratio of 1:1 or 1:0.2. CAR-T cells co-cultured with CD19-expressing target tumour cells (NALM6) were included as positive control. After 48 hours, the frequency of T cells that had gone through cell division was analyzed using flow cytometry. T cells cultured alone (T cells alone) were included as a control for background proliferation. A) Representative flow cytometry plot showing the CAR T cell proliferation gating based on CellTrace dye intensity after 48-hour co-culture. B) Bar plot showing the frequency of anti-CD19 CAR T cells that entered cell division after 48-hour co-culture stimulation. Exemplary data from one allogeneic donor experiment. The experiment was performed using technical duplicates.

FIG. 10—CAR T cell exhaustion and anti-tumour cytotoxicity after U-PDCENHANCE co-culture. Anti-CD19-CAR T cells were co-cultured with Mock U-pDCs, tCD19-U-pDCs^{ENHANCE}, or tCD19-K562 tumour cells at a T cell: Enhance cell ratio of 1:0.2. Mock T cell co-cultures were included as negative control. After 24 hours of pre-conditioning, co-culture T cells were isolated using a commercial immunomagnetic negative selection kit. The purified CAR-T cells were subsequently seeded at 2,000 cells per well and challenged with 16,000 tumour cells (NALM6). The CAR T cell cultures were rechallenged by addition 16,000 NALM6 cells every 48-72 hours for a total of three challenges. The tumour cell expansion was analyzed 48-72 hours post tumour challenge by flow cytometry. A) Graph showing the tumour survival/expansion during three cumulative tumour challenges of pre-conditioned anti-CD19 CAR T cells. B) Bar plot showing the expression of T cell exhaustion markers after three cumulative tumour challenges of pre-conditioned anti-CD19 CAR T cells. Exemplary data from one allogeneic donor experiment. The experiment was performed using technical triplicates.

FIG. 11—Endogenously expressed co-stimulatory receptors on UpDC^{ENHANCE} cells aid CAR T activation. UpDC^{ENHANCE} (A) or NALM6 tumor cells control (B) were pre-incubated with a neutralizing antibody cocktail against co-stimulatory receptors: CD40, CD80, CD86, and ICAM-1 (a-co-recept.), antibody isotype controls (Isotype ctrl.) or left untreated (UT). After 5 h, Anti-CD19 CAR T cells (allogeneic) were added at a T cell to ENHANCE cell ratio of 1:0.2 (A) or 1:1 (B). Culture supernatants were harvested after 48 h and the level of T cell activation associated cytokines IFN-y and IL-2 was quantified by ELISA. All graphs show the mean+SD of duplicate co-cultures.

FIG. 12—CAR T cell and UpDCs^{ENHANCE} interaction leads to increased secretion of CXCL10. Anti-CD19 CAR T cells were co-cultured with Mock UpDCs, UpDC^{ENHANCE}, or NALM6 in 1:1 ratio (allogeneic). Supernatants were collected after 48 h and CXCL10 levels were quantified by Simple Plex Automated ELISA (ELLA, BioTechne). Bar graph shows the mean+SD cytokine response subtracted background response. The data represents three experiments (UpDC=1× CAR T donors=3). Statistical analysis was performed using One-way ANOVA with Tukey's multiple-comparisons test. * p<0.0332, ** p<0.0021, *** p<0.0002, **** p<0.0001.

FIG. 13—CAR T cell expansion after UpDCs^{ENHANCE} boost in vivo. A) Overview figure describing experimental setups to boost CAR T expansion by UpDC^{ENHANCE} administration in the context of either non-tumor bearing (top panel) or tumor bearing mice (lower panel). CAR T cells (5×10\hat{ }6) were intravenously (i.v.) injected into NXG [NOD-Prkdc^{scid}_IL2rg^{Tm1}/Rj] mice. After 24 h the mice were injected (i.v.) with either 5×10\hat{ }6UpDC^{ENHANCE} cells (allogeneic) or PBS. The spleens were harvested and analyzed for CAR T frequency 5 (top panel) or 7 (lower panel) days after the CAR T injection. A) Bar graph showing the percentage of CD8⁺ T cells out of total live cells found in spleen of UpDCs^{ENHANCE}-(n=2) vs PBS-treated (n=1) non-tumor bearing mice as described in (A). The spleens were harvested 5 days after the CAR T injection and dissociated into single cell suspension. The percentage of CD8⁺ CAR T cells was determined by flow cytometry. CD8⁺ cells were pre-gated on: Single cells, Live, Ter119^{neg}, CD45Pos cells. B) Bar graph showing the percentage of CD8⁺ T cells out of total live cells in spleens of UpDCs^{ENHANCE}-(n=3) vs PBS-treated (n=2) tumor-bearing mice as described in (A). Seven days after the CAR T cell injection, the spleens of the mice and dissociated into single cell suspension. The percentage of CD8⁺ T cells was determined by flow cytometry. CD8⁺ cells were pre-gated on: Live, Ter119^{neg}, NALM6-Katushka^{neg}, CD123^{neg}. All graphs show the mean+SD. Symbols represent individual mice.

FIG. 14—UpDCs^{ENHANCE} boost CAR T anti-tumor cytotoxicity in vivo. A sub-optimal dose of anti-CD19 CAR T cells (5×10\hat{ }6 cells) was injected (i.v.) into NXG [NOD-Prkdc^{scid}_IL2rg^{Tm1}/Rj] mice with established NALM6 flank tumors (105-263mm³). After 24 h the mice were injected with either 1×10\hat{ }6 UpDC^{ENHANCE} cells (allogeneic) or PBS (n=4 per group). After seven days, the expansion in tumor volume (A), and the endpoint tumor weight (B) was determined. All graphs show the mean+SD. Symbols represent individual mice.

FIG. 15. UpDCs can be modified to express a different CAR-antigen. The experiment for which results are reported in FIG. 5B was expanded to also include transduction with HER2. Bar graph showing the transduction efficacy of tCD19- and tHER2-UpDC^{ENHANCE}. tCD19-UpDCs were generated from seven donors (buffy coat (n=3) or cord-blood derived CD34⁺ HSPCs (n=4)), while tHER2-UpDC^{ENHANCE} were generated from three cord-blood derived CD34⁺ HSPC donors. The transduction efficiency was determined by flow cytometry staining and gated after a non-transduced control as in A. The bars show the mean and standard deviation (SD) of individual UpDC donors (represented by symbols).

FIG. 16. Expansion and immunophenotype of U-pDCs genetically modified to express truncated CD19. The experiment as described in FIG. 6B was repeated to incorporate technical replicates and statistical analysis. Bar plot showing the frequency of cord-blood derived IFN-primed Mock-and tCD19-U-pDCs^{ENHANCE} (n=5) with pDC-marker expression (CD123 and CD303). Error bars show SD. Statistical analysis was performed using Two-way ANOVA with Šídak's multiple comparisons test. * p<0.0332, ** p<0.0021, *** p<0.0002, **** p<0.0001.

FIG. 17. Type I IFN responses of U-pDCs genetically modified to express truncated CD19. The experiment as set out in FIG. 7 was repeated, using primed or unprimed U-pDCs where 71-95% of the U-pDC^{ENHANCE} cells express a truncated CD19. Stimulation was performed in technical duplicates (UpDC batches: n=5). Error bars show SD. Symbols represent individual UpDC donors. Statistical analysis was performed using Two-way ANOVA with Tukey's multiple comparisons test. * p<0.0332, ** p<0.0021, *** p<0.0002, **** p<0.0001.

FIG. 18. UpDCSENHANCE boost CAR T cell activation in vitro. CAR T cells were co-cultured with IFN-primed Mock UpDCs or UpDC^{ENHANCE} in technical duplicates. CAR T cells co-cultured with target tumor cells were included as positive activation control. After 48 hours, supernatants were harvested and secretion of T cell activation associated cytokines (IL-2, TFNα, and IFNyγ) was quantified by commercially available ELISA. A) Bar graph showing the secreted IL-2, TFNα, and IFNγ levels from anti-CD19 CAR T cells co-cultured (allogeneic) with Mock UpDCs, UpDC^{ENHANCE}, or target tumor cells (tCD19-K562) at the indicated co-culture ratios at a T cell: ENHANCE cell ratio of 1:1. Statistical analysis was performed using Two-way ANOVA with Šídák's multiple comparisons test. B-C) Bar plots showing the secreted IL-2, TFNα, and IFNγ levels from anti-CD19 (B) or anti-HER2 (C) CAR T cells co-cultured (allogeneic) with Mock UpDCs, UpDC^{ENHANCE}, or target tumor cells (NALM6 or tHER2-NALM6) at the indicated co-culture ratios. The anti-CD19 CAR T co-culture plot (B) shows the average of six replicate experiments (UpDC donors=2× CAR T donors=3), the anti-HER2CAR T co-culture plot (C) shows the average of three experiments (UpDC donor=1× CAR T donors=3). Statistical analysis was performed using Two-way ANOVA with Dunnett's multiple comparisons test. All co-cultures were performed in technical duplicates. Error bars show SD. * p<0.0332, ** p<0.0021, *** p<0.0002, **** p<0.0001.

FIG. 19—UpDCs^{ENHANCE} increases CAR T cell proliferation in vitro. CAR T cells were stained with CellTrace Yellow dye and co-cultured with Mock UpDCs or UpDC^{ENHANCE} in technical duplicates. The CAR T cells co-cultured with tumor cells (NALM6) were used as a positive control. After 120 hours, the cells were harvested and analyzed for proliferation by flow cytometry. A) Example histogram of the decrease in CellTrace Yellow fluorescence intensity as a result of cell division. The plot shows CAR T proliferation after autologous Mock UpDC and tCD19-UpDC^{ENHANCE} co-cultures compared with co-culture with target tumor cells (NALM6, allogeneic) at CAR T to EHANCE cell ratios of 1:0.2. B) Bar graph showing the allogeneic and autologous anti-CD19 CAR T expansion index (fold cell expansion) 120 h post co-culture with Mock UpDCs, UpDC^{ENHANCE}, or target tumor cells (NALM6, allogeneic) at the indicated co-culture ratios. Indexes were calculated using proliferation modelling in FlowJo V.10.8.1. The allogeneic plot shows the average of six replicate experiments (UpDC donors=2× CAR T donors=3), while the autologous plot shows the results of one donor pair. C) Bar graph showing anti-HER2 CAR T (allogeneic) proliferation index as described in (B). Here HER2-NALM6 cells were used as positive control. The plot shows the average of three experiments (UpDC donor=1 x CAR T donors=3). Error bars show SD. Statistical analysis was performed using One-way ANOVA with Dunnett's multiple comparisons test. * p<0.0332, ** p<0.0021, *** p<0.0002, **** p<0.0001.

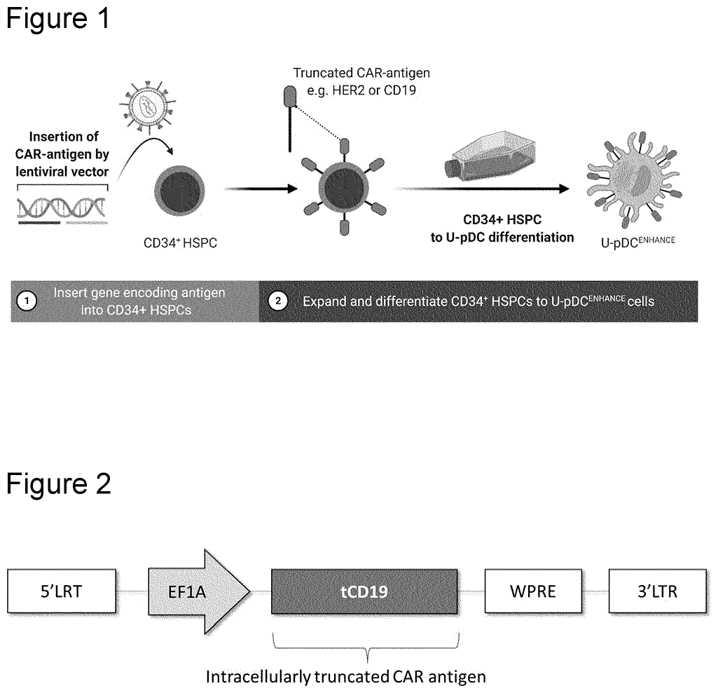

### DETAILED DESCRIPTION OF THE INVENTION

### Plasmacytoid Dendritic Cells (pDCs) for Treating Disease

The engineered cells for use in the invention are plasmacytoid dendritic cells (pDCs). Plasmacytoid dendritic cells (pDCs) have a multifaceted role in the immune system, which makes them extremely adaptable for the targeted treatments of the invention. pDCs are key effectors in cellular immunity with the ability to not only initiate immune responses but also to induce tolerance to exogenous and endogenous antigens (Swiecki, and Colonna, Nat Rev Immunol, 2015. 15(8)). pDCs are distinct from conventional DCs as their final stage of development occurs within the bone marrow; their antigens are taken up by receptor-mediated endocytosis; they express high levels of interferon regulatory factor 7; and they primarily sense pathogens through toll-like receptor (TLR) 7 and 9 (Swiecki and Colonna, Nat Rev Immunol, 2015. 15(8); and Tangand Cattral, Cell Mol Life Sci, 2016). Through these pattern-recognition receptors, pathogen nucleic acids can activate pDCs to produce high levels of type I interferon (IFN). Thus, activated pDCs link the innate and adaptive immune system together via cytokine production combined with antigen-presenting cell (APC) activity. Furthermore, pDC functionality is also essential to achieve an antiviral state during infections, provide vital adjuvant activity in the context of vaccination, and for promoting immunogenic anti-tumour responses upon activation (Swiecki, and Colonna, Nat Rev Immunol, 2015. 15(8); Tovey, et al. Biol Chem, 2008. 389(5); and Rajagopal, et al. Blood, 2010. 115(10): p. 1949-57). A delicate balance must be maintained, however, as hyper-activation of pDCs has been associated with the pathogenesis of several diseases, including viral infections, autoimmune diseases and tumourigenesis (Swiecki and Colonna, Nat Rev Immunol, 2015. 15(8); and Tang and Cattral, Cell Mol Life Sci, 2016).

The pDCs for use in the invention express at least one antigen that is bound by a receptor expressed by the adoptive cell transfer immunotherapy, or the pDCs express at least one receptor that binds an antigen expressed by the adoptive cell transfer immunotherapy. The antigen or receptor expressed by the pDCs is preferably presented on the surface.

In certain embodiments, the antigen is expressed in a MHC complex. In certain embodiments, the antigen is expressed as part of a single chain MHC peptide complex. Such complexes are known to be effective for presenting antigens (as reported in, for example, Kotsiou et al., 2011, Antioxid Redox Signal, 15(3): 645-55). pDCs presenting an antigen in a MHC complex, such as in a single chain MHC peptide complex, are expected to be particularly effective for stimulating TCR-engineered T-cells, and to enhance their known effectiveness for treating diseases (as reported in, for example, Shafer et al., 2022, Front. Immunol. 13:835762. doi: 10.3389/fimmu.2022.835762). In certain embodiments the MHC complex comprises an invariant chain. In certain embodiments the MHC complex comprises MR1. In certain embodiments the MHC complex is MHC class II. In certain embodiments, the antigen expressed by the pDC and bound by the ACT is selected to mediate a graft v leukemia effect, for example the antigen may be HA-1, ACC-1, ACC-2, and LRH1, described below.

In certain embodiments, the antigen is expressed without a MHC complex, for example using appropriate transmembrane domains and/or targeting peptides. pDCs expressing antigens on their surface are expected to be effective for stimulating a range of ACT cells, as demonstrated in the examples.

In one embodiment, the engineered pDCs express TRAIL. In another embodiment said pDCs express CD123, CD303, CD304, CD4 and/or HLA-DR. In yet another embodiment said pDCs express IFN type I, IFN type III and/or proinflammatory cytokines. In a further embodiment said pDCs express IRF7, TLR7 and/or TLR9. In one embodiment said pDCs express CD40, CD80, CD83 and/or CD86. For example, said pDCs express TRAIL, CD123, CD303, CD304, CD4, HLA-DR, IFN type I, IFN type III, IRF7, TLR7 and/or TLR9.

The engineered pDCs of the invention are preferably matured cells having a surface phenotype that strongly resembles blood pDCs. In a preferred embodiment, said pDCs express CD123, CD303, CD304, CD4 and/or HLA-DR.

In another preferred embodiment said pDCs express IFN type I, IFN type III and/or proinflammatory cytokines.

The pDCs may in one preferred embodiment express Toll-like receptors, such as for example Toll-like receptor 7 (TLR7) and/or Toll-like receptor 9 (TLR9).

In yet another preferred embodiment said pDCs express Interferon regulatory factor 7 (IRF7).

In a preferred embodiment said pDCs secretes IL-6.

In preferred embodiments, the engineered plasmacytoid dendritic cell is capable of a type I IFN response. In another preferred embodiment said pDCs express Cluster of differentiation 80 (CD80), which is a protein found on Dendritic cells, activated B cells and monocytes that provides a costimulatory signal necessary for T cell activation and survival.

The pDCs may in a preferred embodiment also express proteins characteristic for antigen presenting cells such as for example Cluster of Differentiation 86 (CD86) and/or Cluster of Differentiation 40 (CD40). CD86 is a protein expressed on antigen-presenting cells that provides costimulatory signals necessary for T cell activation and survival, whereas CD40 is a costimulatory protein found on antigen presenting cells and is required for their activation.

Preferably, said pDCs express CD40, CD80, CD83 and/or CD86. In another preferred embodiment said pDCs express interleukin 6 (IL-6). In a preferred embodiment, the pDCs are immunogenic. In preferred embodiments, the pDCs stimulate an immune response against the antigen. In some embodiments, the pDCs secrete CXCL10, or promote CXCL10 secretion.

In preferred embodiments, the engineered pDCs of the invention are stem cell-derived plasmacytoid dendritic cell.

In certain embodiments, pDCs are autologous. Such treatments may minimise any risk of rejection of the transferred cells. In alternative embodiments, the cells are allogenic, such as isolated from healthy donors. Such treatments can potentially be prepared more quickly and offered “off the shelf”. In certain embodiments, the cells are or have been cryopreserved. Moreover, the cells may be xenogeneic.

### Adoptive Cell Transfer Immunotherapies

The methods of the invention provide increased benefits from adoptive cell transfer (ACT) immunotherapies and thereby provide improved methods of treating cancer, autoimmune conditions, inflammatory diseases and infectious diseases. ACT immunotherapies are an established and potent approach for treating cancer in particular. ACT is the passive transfer of ex vivo grown cells, most commonly immune-derived cells, into a host with the goal of transferring the immunologic functionality and characteristics of the transplant.

ACT can be autologous (e.g., isolated by leukapheresis, transduced and selected approximately 4 weeks immediately prior to administration), as is common in adoptive T-cell therapies, or allogeneic, in which case the methods of the invention may improve the ACT by removing antibodies that recognise the expressed receptor and/or other antigens on the allogenic cells. Moreover, the ACT may be xenogeneic. In preferred embodiments, ACT is autologous.

ACT may also comprise transfer of autologous tumour infiltrating lymphocytes (TILs) which may be used to treat patients with advanced solid tumours such as melanoma and hematologic malignancies.

ACT may also comprise transfer of allogeneic lymphocytes isolated, prepared, and stored (e.g., frozen) “off-the-shelf” from a healthy donor which may be used to treat patients with advanced solid tumours, such as melanoma, and hematologic malignancies.

The adoptive cell immunotherapy of the invention may include administration of cells expressing a chimeric antigen receptor (CAR), or a T-cell receptor (TCR), or may include tumour-infiltrating lymphocytes (TIL). The population of cells expressing the CAR/TCR, which recognize an antigen, may comprise a population of activated T-cells, natural killer (NK) cells, macrophages or dendritic cells. Dendritic cells are capable of antigen presentation, as well as direct killing of tumours. Dendritic cells may express, for example, 4-1BB or an anti-CD19 CAR. CAR macrophages are also known to be effective (for example as reported in Klichinsky et al. Nat Biotechnol. 2020 August; 38(8): 947-953). The population of cells expressing the CAR/TCR may comprise a population of gene-edited cells.

The ACT may use cell types such as T-cells, natural killer (NK) cells, delta-gamma T-cells, regulatory T-cells, dendritic cells, macrophages and peripheral blood mononuclear cells. The ACT may use monocytes with the purpose of inducing differentiation to dendritic cells and/or macrophages subsequent to contact with tumour antigens.

According to preferred embodiments of the invention, the adoptive cell therapy may be a CAR T-cell therapy. The CAR T-cell can be engineered to target a tumour antigen of interest by way of engineering a desired antigen binding domain that specifically binds to an antigen on a tumour cell. In preferred embodiments, the cell therapy uses a cell of hematopoietic origin. The examples demonstrate that the methods of the invention are particularly effective against cells of hematopoietic origin.

In preferred embodiments, the CAR T-cell therapy employs CAR T-cells that target CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPA7, PSCA, EGFR, EGFRVIII, ROR1, mesothelin, CD33/IL3Ra, c-Met, CD37, PSMA, Glycolipid F77, GD-2, gp100, NY-ESO-1 TCR, FRalpha, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or a combination thereof (e.g., both CD33 and CD123). Preferred antigens are BCMA, CD19, CD20 and CD22.

The ACT immunotherapy may be a CAR T-cell therapy (e.g., autologous cell therapy and allogeneic cell therapy). A CAR T-cell therapy may be suitable for treating hematologic malignancies such as ALL, AML, NHL, DLBCL and CLL. Examples of approved CAR T-cell therapies include, without limitation, KYMRIAH® (tisagenlecleucel) for treating NHL and DLBCL, and YESCARTA® (axicabtagene ciloleucel) for treating NHL. A CAR T-cell therapy may be suitable for treating solid tumours. In preferred embodiments, the cancer is a solid tumour. The pDCs of the invention may be particularly effective at improving the ability of an ACT immunotherapy to survive in the microenvironment of solid tumours.

The ACT may be a CAR-NK or a CAR-macrophage. CAR-NK and CAR-macrophages therapies are effective for treating solid tumours and hematologic malignancies and their effectiveness is expected to be improved upon stimulation with pDCs according to the invention.

According to certain aspects of the present invention, the population of cells expressing the CAR/TCR or the TIL may be autologous cells, allogeneic cells derived from another human donor, or xenogeneic cells derived from an animal of a different species.

According to certain aspects of the present invention, the population of cells expressing the CAR/TCR or the TIL may be isolated by leukapheresis, transduced and selected approximately 4 weeks immediately prior to administration, as in the case of autologous stem cells, or may be isolated from a healthy donor and prepared in advance then stored, such as a frozen preparation, for one or more patients as in the case of so called “off-the-shelf” allogeneic CAR-T stem cell therapies.

According to certain aspects of the present invention, the population of cells expressing the CAR/TCR may comprise a population of activated T-cells or natural killer (NK) cells or macrophages or dendritic cells expressing the CAR/TCR which recognize an antigen. Dendritic cells are capable of antigen presentation, as well as direct killing of tumours.

According to certain aspects of the present invention, the antigen may be one that is expressed only on cancer cells or one that is preferentially expressed on cancer cells, such as a neo-antigen or lineage-specific antigen (such as CD19 or CD20).

The CAR T-cell may comprise an antigen binding domain capable of targeting two or more different antigens (i.e., bispecific or bivalent, trispecific or trivalent, tetraspecific, etc.). As such, the CAR T-cell may comprise a first antigen binding domain that binds to a first antigen and a second antigen binding domain that binds to a second antigen (e.g., tandem CAR). For example, the CAR T-cell may comprise a CD19 binding domain and a CD22 binding domain and may thus recognize and bind to both CD19 and CD22. Or further, the CAR T-cell may comprise a CD19 binding domain and a CD20 binding domain and may thus recognize and bind to both CD19 and CD20.

Alternatively, each cell in the population of cells, or the overall population of cells, may comprise more than one distinct CAR T-cell (e.g., construct), wherein each CAR T-cell construct may recognize a different antigen. For example, the population of CAR T-cells may target three antigens such as, for example, HER2, IL13Ra2, and EphA2.

According to certain aspects of the present invention, the population of cells, whether autologous or allogeneic, may be engineered using gene editing technology such as by CRISPR/cas9 (clustered regularly interspaced short palindromic repeats/CRISPR associated protein 9), Zinc Finger Nucleases (ZFN), or transcription activator-like effector nuclease (TALEN). These technologies, recognized and practiced in the art of genetic engineering, enable selective editing, disruption, or insertion of targeted sequences to modify the genome of the cell of interest. Accordingly, isolated autologous or allogeneic cells for adoptive transfer practiced in the current invention may be edited to delete or replace a known gene or sequence. For example, the T cell receptor (TCR) in an allogeneic T cell population may be deleted or replaced prior to or after CAR-T transduction as a means to eliminate graft-versus-host disease in recipient patients.

According to certain aspects of the present invention, the population of cells administered as the ACT immunotherapy may comprise a population of T-cells, NK-cells, macrophages or dendritic cells expressing a CAR, wherein the CAR comprises an extracellular antibody or antibody fragment that includes a humanized anti-CD19 binding domain, a humanized anti-CD22 binding domain, a humanized anti-CD20 binding domain or a humanized anti-BCMA binding domain, a transmembrane domain, and one or more cytoplasmic co-stimulatory signalling domains. The population of cells may comprise a population of cells expressing a CAR, wherein the CAR comprises an extracellular antibody or antibody fragment that includes two or more binding domains, such as a humanized anti-CD19 binding domain, a humanized anti-CD22 binding domain, a humanized anti-CD20 binding domain, and/or a humanized anti-BCMA binding domain, and a transmembrane domain and one or more cytoplasmic co-stimulatory signalling domains.

In certain embodiments of the invention, the population of cells administered as the ACT immunotherapy express T-cell receptors (TCRs). TCRs are antigen-specific molecules that are responsible for recognizing antigenic peptides presented in the context of a product of the major histocompatibility complex (MHC) on the surface of antigen presenting cells or any nucleated cell (e.g., all human cells in the body, except red blood cells). In contrast, antibodies typically recognize soluble or cell-surface antigens, and do not require presentation of the antigen by an MHC. This system endows T-cells, via their TCRs, with the potential ability to recognize the entire array of intracellular antigens expressed by a cell (including virus proteins) that are processed intracellularly into short peptides, bound to an intracellular MHC molecule, and delivered to the surface as a peptide-MHC complex. This system allows virtually any foreign protein (e.g., mutated cancer antigen or virus protein) or aberrantly expressed protein to serve as a target for T-cells.

In further embodiments, the method uses a T-cell that targets a B-cell population that produces deleterious antibodies. In certain embodiments, the cell used is a chimeric autoantibody receptor T (CAAR-T) cell. In such embodiments, the cell expresses a construct presenting an antigen that is recognised by the problematic B-cells, such as a drug antigen, or an auto-antigen, and the B-cell is eliminated upon binding the therapeutic cell. Such cells are also useful for treating autoimmune conditions and other conditions caused by B-cells producing deleterious antibodies.

In further embodiments, the method uses an ACT that targets a fibroblast population associated with disease, such as fibrosis (as reported in, for example, Aghajanian et al. Nature. 2019 573(7774): 430-433). Accordingly, in certain embodiments of any aspect of the invention, the method of the invention is for treating fibrosis.

According to certain aspects of the present invention, the engineered CAR cell may be allogeneic from a healthy donor and be further engineered to ablate or replace the endogenous TCR by gene editing technology such as CRISPR/cas9, ZFN, or TALEN, wherein the deletion of the endogenous TCR serves to eliminate CAR driven graft-versus-host disease.

According to certain aspects of the present invention, autologous cells (e.g., T-cell or NK-cells or macrophages or dendritic cells) may be collected from the subject. These cells may be obtained from a number of sources, including peripheral blood mononuclear cells, bone marrow, lymph node tissue, cord blood, thymus tissue, tissue from a site of infection, ascites, pleural effusion, spleen tissue, and tumours. According to certain aspects of the present invention, allogeneic or xenogeneic cells may be used, typically isolated from healthy donors. When the T-cells, NK cells, dendritic cells, macrophages or pluripotent stem cells are allogeneic or xenogeneic cells, any number of cell lines available in the art may be used.

The cells can be obtained from a unit of blood collected from a subject using any number of techniques known to the skilled artisan, such as Ficoll™ separation. According to certain aspects of the present invention, cells from the circulating blood of an individual may be obtained by apheresis. The apheresis product typically contains lymphocytes, including T-cells, B-cells, monocytes, granulocytes, other nucleated white blood cells, red blood cells, and platelets.

Enrichment of a cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1 1b, CD16, HLA-DR, and CD8. According to certain aspects of the present invention, it may be desirable to enrich for or positively select for a cell population. For example, positive enrichment for a regulatory T-cell may use positive selection for CD4+, CD25+, CD62Lhi, GITR+, and FoxP3+.

The collected cells may be engineered to express the CAR or TCR by any of a number of methods known in the art. Moreover, the engineered cells may be expanded by any of a number of methods known in the art. As detailed above, the CAR or TCR may be bispecific, trispecific, or quadraspecific; the CAR or TCR may include a switch such as a goCAR or goTCR, or a safety switch CAR or TCR; the CAR or TCR may express immune-modulatory proteins such as an armored CAR or TCR.

According to certain aspects of the present invention, the collection of blood samples or apheresis product from a subject may be at any time period prior to when the expanded cells as described herein might be needed. As such, the source of the cells to be engineered and expanded (or simply expanded in the case of TILs) can be collected at any time point necessary, and desired cells, such as T-cells, NK-cells, macrophages, dendritic cells, or TILs, can be isolated and frozen for later use in ACT, such as those ACT described herein.

According to certain aspects of the present invention, the population of cells expressing the CAR/TCR may be administered to the subject by dose fractionation, wherein a first percentage of a total dose is administered on a first day of treatment, a second percentage of the total dose is administered on a subsequent day of treatment, and optionally, a third percentage of the total dose is administered on a yet subsequent day of treatment.

An exemplary total dose comprises 10³ to 10¹¹ cells/kg body weight of the subject, such as 10³ to 10¹⁰ cells/kg body weight, or 10³ to 10⁹ cells/kg body weight of the subject, or 10³ to 10⁸ cells/kg body weight of the subject, or 10³ to 10⁷ cells/kg body weight of the subject, or 10³ to 10⁶ cells/kg body weight of the subject, or 10³ to 10⁵ cells/kg body weight of the subject. Moreover, an exemplary total dose comprises 10⁴ to 10¹¹ cells/kg body weight of the subject, such as 10⁵ to 10¹¹ cells/kg body weight, or 10⁶ to 10¹¹ cells/kg body weight of the subject, or 10⁷ to 10¹¹ cells/kg body weight of the subject.

An exemplary total dose may be administered based on a patient body surface area rather than the body weight. As such, the total dose may include 10³ to 10¹³ cells per m².

An exemplary dose may be based on a flat or fixed dosing schedule rather than on body weight or body surface area. Flat-fixed dosing may avoid potential dose calculation mistakes. Additionally, genotyping and phenotyping strategies, and therapeutic drug monitoring, may be used to calculate the proper dose. That is, dosing may be based on a patient's immune repertoire of immunosuppressive cells (e.g., regulatory T cells, myeloid-derived suppressor cells), and/or disease burden. As such, the total dose may include 10³ to 10¹³ total cells.

According to certain aspects of the present invention, cells may be obtained from a subject directly following a treatment. In this regard, it has been observed that following certain cancer treatments, in particular treatments with drugs that damage the immune system, shortly after treatment during the period when subjects would normally be recovering from the treatment, the quality of certain cells (e.g., T-cells) obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T-cells, NK-cells, dendritic cells, macrophages or other cells of the hematopoietic lineage, during this recovery phase.

According to certain aspects of the present invention, the second dose may be the same or a different effective amount of a different population of cells expressing the same or a different CAR/TCR. Differences in the CAR/TCR may be in any aspect of the CAR/TCR such as, for example, different binding or antigen recognition domains or co-stimulatory domains. The second dose may additionally or alternatively include secreting cells with IL-12 or may even include adjuvant immunotherapies with small molecule inhibitors such as BTK, P13K, IDO inhibitors either concurrent or sequential to the cell therapy infusion.

According to certain aspects of the present invention, the methods may also comprise administration of one or more additional therapeutic agents, in addition to the ACT immunotherapy and the pDCs. Exemplary therapeutic agents include a chemotherapeutic agent, an anti-inflammatory agent, an immunosuppressive, an immunomodulatory agent, or a combination thereof.

Therapeutic agents may be administered according to any standard dose regime known in the field. Exemplary chemotherapeutic agents include anti-mitotic agent, such as taxanes, for instance docetaxel, and paclitaxel, and vinca alkaloids, for instance vindesine, vincristine, vinblastine, and vinorelbine. Exemplary chemotherapeutic agents include a topoisomerase inhibitor, such as topotecan.

Exemplary chemotherapeutic agents include a growth factor inhibitor, a tyrosine kinase inhibitor, a histone deacetylase inhibitor, a P38a MAP kinase inhibitor, inhibitors of angiogenesis, neovascularization, and/or other vascularization, a colony stimulating factor, an erythropoietic agent, an anti-anergic agents, an immunosuppressive and/or immunomodulatory agent, a virus, viral proteins, immune checkpoint inhibitors, BCR inhibitors (e.g., BTK, P13K, etc.), immune-metabolic agents (e.g., IDO, arginase, glutaminase inhibitors, etc.), and the like. According to certain aspects of the present invention, the one or more therapeutic agents may comprise an antimyeloma agent. Exemplary antimyeloma agents include dexamethasone, melphalan, doxorubicin, bortezomib, lenalidomide, prednisone, carmustine, etoposide, cisplatin, vincristine, cyclophosphamide, and thalidomide, several of which are indicated above as chemotherapeutic agents, anti-inflammatory agents, or immunosuppressive agents.

### Methods for Treating Cancer

The invention provides methods of treating cancer comprising administering pDCs in combination with an ACT immunotherapy, wherein the pDCs express at least one antigen that is bound by a receptor expressed by the ACT immunotherapy. Therefore, the invention provides a new ACT immunotherapy. ACT immunotherapy is the transfer of ex vivo grown cells, most commonly immune-derived cells, into a host with the goal of transferring the immunologic functionality and characteristics of the transferred cells. ACT immunotherapy is well established for treating cancer and autoimmune, inflammatory and infectious diseases.

The ability of pDCs to improve the activity, cytokine production and proliferation of an ACT immunotherapy and to reduce exhaustion on an ACT immunotherapy are expected to be particularly useful for treating cancer.

In certain embodiments of the invention, the ACT immunotherapy comprises T cells, natural killer cells or dendritic cells. In some embodiments, the ACT immunotherapy comprises cells expressing a chimeric antigen receptor (CAR), a T-cell receptor, or a synthetic Notch receptor.

In certain embodiments of the invention, the methods of treatment may comprise (i) collecting autologous hematopoietic stem progenitor cells (HSPCs), either from the subject to be treated or a healthy donor; (ii) preparing engineered pDCs, for example using a method discussed below; (iii) optionally administering to the subject lymphodepleting chemotherapy; and (iv) administering to the subject the engineered pDCs in combination with an ACT immunotherapy.

In certain embodiments, the methods of the invention may comprise administering pDCs expressing more than one antigen. Individual cells may express more than one antigen, or the population of cells administered may comprise a plurality of different cells.

In certain embodiments, the engineered cells are further modified to express immune-modulatory proteins, such as cytokines (e.g., IL-2, IL-12 or IL-15), which may stimulate T-cell activation and recruitment, and may thus aid in combating the tumour microenvironment. Thus, the cells may comprise a population of cells expressing the exogenous construct and further expressing an immune modulatory protein such as, for example, IL-2, IL-12, or IL-15.

In certain embodiments, the engineered cells may be isolated from a subject and used fresh, or frozen for later use, in conjunction with (e.g., before, simultaneously or following) lymphodepletion.

In certain embodiments, the pDCs are administered simultaneously with the ACT immunotherapy. In certain embodiments, the pDCs are administered separately from the ACT immunotherapy. In some embodiments, the pDCs are administered prior to the ACT immunotherapy. In some embodiments, the pDCs are administered subsequent to the ACT immunotherapy. In some embodiments, the pDCs are administered repeatedly in two or more doses subsequent to the ACT immunotherapy.

In certain embodiments, the engineered cells may be administered to the subject by dose fractionation, wherein a first percentage of a total dose is administered on a first day of treatment, a second percentage of the total dose is administered on a subsequent day of treatment, and optionally, a third percentage of the total dose is administered on a yet subsequent day of treatment.

An exemplary total dose comprises 10³ to 10¹¹ cells/kg body weight of the subject, such as 10³ to 10¹⁰ cells/kg body weight, or 10³ to 10⁹ cells/kg body weight of the subject, or 10³ to 10⁸ cells/kg body weight of the subject, or 10³ to 10⁷ cells/kg body weight of the subject, or 10³ to 10⁶ cells/kg body weight of the subject, or 10³ to 10⁵ cells/kg body weight of the subject. Moreover, an exemplary total dose comprises 10⁴ to 10¹¹ cells/kg body weight of the subject, such as 10⁵ to 10¹¹ cells/kg body weight, or 10⁶ to 10¹¹ cells/kg body weight of the subject, or 10⁷ to 10¹¹ cells/kg body weight of the subject.

An exemplary total dose may be administered based on a patient body surface area rather than the body weight. As such, the total dose may include 10³ to 10¹³ cells per m².

In certain embodiments of the invention, the methods comprise lymphodepletion. Lymphodepletion may be achieved by any appropriate means. Lymphodepletion may be performed prior to administration of the engineered cells, or subsequent to. In certain embodiments, lymphodepletion is performed both before and after administration of the engineered cells.

In certain embodiments of the invention, the methods may comprise administration of one or more additional therapeutic agents. Exemplary therapeutic agents include a chemotherapeutic agent, an anti-inflammatory agent, an immunosuppressive, an immunomodulatory agent, or a combination thereof.

Therapeutic agents may be administered according to any standard dose regime known in the field. Exemplary chemotherapeutic agents include anti-mitotic agent, such as taxanes, for instance docetaxel, and paclitaxel, and vinca alkaloids, for instance vindesine, vincristine, vinblastine, and vinorelbine. Exemplary chemotherapeutic agents include a topoisomerase inhibitor, such as topotecan. Exemplary chemotherapeutic agents include a growth factor inhibitor, a tyrosine kinase inhibitor, a histone deacetylase inhibitor, a P38a MAP kinase inhibitor, inhibitors of angiogenesis, neovascularization, and/or other vascularization, a colony stimulating factor, an erythropoietic agent, an anti-anergic agents, an immunosuppressive and/or immunomodulatory agent, a virus, viral proteins, immune checkpoint inhibitors, BCR inhibitors (e.g., BTK, P13K, etc.), immune-metabolic agents (e.g., IDO, arginase, glutaminase inhibitors, etc.), and the like. According to certain aspects of the present invention, the one or more therapeutic agents may comprise an antimyeloma agent. Exemplary antimyeloma agents include dexamethasone, melphalan, doxorubicin, bortezomib, lenalidomide, prednisone, carmustine, etoposide, cisplatin, vincristine, cyclophosphamide, and thalidomide, several of which are indicated above as chemotherapeutic agents, anti-inflammatory agents, or immunosuppressive agents.

Treatment of cancer, according to the invention, refers to a biological effect that may present as a decrease in disease burden, disease incidence or disease severity. For example, this may manifest as a reduction in tumour volume, a decrease in the number of tumour cells, a decrease in tumour cell proliferation, a decrease in the number of metastases, an increase in overall or progression-free survival, an increase in life expectancy, or amelioration of various physiological symptoms associated with the tumour.

The engineered cells of the invention may be administered by any appropriate route. Generally, the cells will be administered by intravenous infusion.

In preferred embodiments, the cancer is a solid tumour. The pDCs of the invention may be particular effective at improving the ability of an ACT immunotherapy to survive in the microenvironment of solid tumours. Accordingly, in certain embodiments, the cancer is a solid cancer, such as a cancer selected from the group consisting of: bone cancer, breast cancer, pancreatic cancer, skin cancer, cancer of the head or neck, cutaneous or intraocular malignant melanoma, uterine cancer, ovarian cancer, prostate cancer, rectal cancer, cancer of the anal region, colon cancer, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, glioblastoma, pituitary adenoma, Kaposi's sarcoma, epidermoid cancer and squamous cell cancer. In some embodiments, treatment reduces tumour volume and/or tumour weight.

In certain embodiments, the method and cells of the invention are for use in treating acute lymphoblastic leukemia (ALL) (including non T cell ALL), acute myeloid leukemia, B cell prolymphocytic leukemia, B-cell acute lymphoid leukemia (“BALL”), blastic plasmacytoid dendritic cell neoplasm, Burkitt s lymphoma, chronic lymphocytic leukemia (CLL), chronic myelogenous leukemia (CML), chronic myeloid leukemia, chronic or acute leukemia, diffuse large B cell lymphoma (DLBCL), follicular lymphoma (FL), hairy cell leukemia, Hodgkin's Disease, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome, non-Hodgkin's lymphoma (NHL), plasma cell proliferative disorder (including asymptomatic myeloma (smoldering multiple myeloma or indolent myeloma), plasmablastic lymphoma, plasmacytoid dendritic cell neoplasm, plasmacytomas (including plasma cell dyscrasia; solitary myeloma; solitary plasmacytoma; extramedullary plasmacytoma; and multiple plasmacytoma), POEMS syndrome (also known as Crow-Fukase syndrome; Takatsuki disease; and PEP syndrome), primary mediastinal large B cell lymphoma (PMBC), small cell-or a large cell-follicular lymphoma, splenic marginal zone lymphoma (SMZL), systemic amyloid light chain amyloidosis, T-cell acute lymphoid leukemia (“TALL”), T-cell lymphoma, transformed follicular lymphoma, or Waldenstrom macroglobulinemia, or a combination thereof. In some embodiments, the cancer is a myeloma. In one particular embodiment, the cancer is multiple myeloma. In some embodiments, the cancer is leukemia. In some embodiments, the cancer is acute myeloid leukemia. In some embodiments, the cancer is relapsed or refractory large B-cell lymphoma, diffuse large B-cell lymphoma (DLBCL) not otherwise specified, primary mediastinal large B-cell lymphoma, high grade B-cell lymphoma, or DLBCL arising from follicular lymphoma.

The antigen expressed by the pDCs is selected from antigens expressed on the surface of the cancer to be treated. In some embodiments, the antigen is selected from the group consisting of CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPA7, PSCA, EGFR, EGFRVIII, ROR1, mesothelin, CD33/IL3Ra, c-Met, CD37, PSMA, Glycolipid F77, GD-2, gp100, NY-ESO-1 TCR, FRalpha, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or a combination thereof (e.g., both CD33 and CD123). Preferred antigens are BCMA, CD19, CD20 and CD22.

In some embodiments, the antigen is selected from the group consisting of 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD70, CD8, CLL-1, c-Met, CMV-specific antigen, CS-1, CSPG4, CTLA-4, DLL3, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumour antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1, HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen, CD38, insulin growth factor (IGF1)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-A1, major histocompatibility complex (MHC) molecule, major histocompatibility complex (MHC) molecule presenting a tumour-specific peptide epitope, M-CSF, melanoma-associated antigen, mesothelin, MN-CA IX, MUC-1, mut hsp70-2, mutated p53, mutated p53, mutated ras, neutrophil elastase, NKG2D, Nkp30, NY-ESO-1, p53, PAP, prostase, prostate specific antigen (PSA), prostate-carcinoma tumour antigen-1 (PCTA-1), prostate-specific antigen protein, STEAP1, STEAP2, PSMA, RAGE-1, ROR1, RU1, RU2 (AS), surface adhesion molecule, survivin, telomerase, TAG-72, the extra domain A (EDA) and extra domain B (EDB) of fibronectin and the Al domain of tenascin-C (TnC Al), thyroglobulin, tumour stromal antigens, vascular endothelial growth factor receptor-2 (VEGFR2), virus-specific surface antigen such as an HIV-specific antigen (such as HIV gpl20), as well as any derivate or variant of these surface markers.

In certain embodiments, the antigen expressed by the pDC and bound by the ACT is selected to mediate a graft v leukemia effect, for example the antigen may be HA-1, ACC-1, ACC-2, and LRH1, described below.

In some embodiments, the antigen is a receptor. In some embodiments, the antigen is modified to disrupt its signalling activity. The signalling activity may be disrupted by truncating the antigen. In some embodiments, the antigen is truncated to remove one or more intracellular signalling domains. In some embodiments, the antigen is truncated to remove one or more intracellular effector domains.

In some embodiments, the methods further comprise administering a chemotherapeutic. In certain embodiments, the chemotherapeutic selected is a lymphodepleting (preconditioning) chemotherapeutic, and is preferably administered before the cells of the invention. Such administration of a chemotherapeutic may improve survival of the transplanted cells.

In the therapeutic methods of the invention, pDCs are administered in combination with an ACT immunotherapy to a subject already suffering from cancer, in an amount sufficient to cure, alleviate or partially arrest the cancer or one or more of its symptoms. Such therapeutic treatment may result in remission, stabilisation, reduction in metastasis or elimination of the cancer. An amount adequate to accomplish this is defined as “therapeutically effective amount”. The subject may have been identified as suffering from cancer and being suitable for an ACT immunotherapy by any suitable means.

In a preferred embodiment, the pDCs administered according to the method are immunogenic. In some embodiments, the pDCs stimulate an immune response against the antigen. In some embodiments, treatment promotes the secretion of CXCL10 within the subject. In some embodiments, the pDCs administered according to the method stimulate the ACT to secrete CXCL10. In some embodiments, the pDCs and/or ACT secrete CXCL10.

### Methods for Treating Autoimmune and Inflammatory Diseases

In preferred embodiments of the invention, the methods and cells of the invention are for use in treating an autoimmune or inflammatory disease. The ability of pDCs to improve the activity, cytokine production and proliferation of an ACT immunotherapy and to reduce exhaustion on an ACT immunotherapy are expected to be particularly useful for treating autoimmune and inflammatory diseases.

In certain embodiments, the autoimmune disease is selected from the list consisting of: type 1 diabetes, thyroid autoimmune diseases (e.g. Hashimoto's and Graves'), Addison's adrenal insufficiency, oophoritis, orchitis, lymphocytic hypophysitis, autoimmune hypoparathyroidism, autoimmune hypoparathyroidism, Goodpasture's disease, autoimmune myocarditis, membranous nephropathy, autoimmune hepatitis, ulcerative colitis, Crohn's disease, multiple sclerosis, myasthenia gravis, neuromyelitis optica, encephalitis and Sjögren's syndrome.

In certain embodiments, the inflammatory disease is selected from the list consisting of: cystic fibrosis, chronic inflammatory intestinal diseases like, for example, ulcerative colitis or Crohn's disease, vasculitis, in particular Kawasaki disease, chronic bronchitis, inflammatory arthritis diseases like, for example, psoriatic arthritis, osteoarthritis, rheumatoid arthritis, and systemic onset juvenile rheumatoid arthritis (SOJRA, Still's disease), graft-versus-host disease, asthma, psoriasis, systemic lupus erythematosus, obesity and inflammatory vascular disease and allograft rejection.

In certain embodiments, the autoimmune or inflammatory disease is transplant rejection or graft-versus-host-disease (GVHD). Exemplary organ transplant to be treated according to the invention include: kidney, heart, lung, liver, intestine, pancreas and islet of Langerhans.

In certain embodiments, the pDCs express at least one receptor that binds an antigen expressed by the ACT immunotherapy and the antigen is recognised by auto-reactive antibodies that cause the autoimmune or inflammatory disease. For example, the ACT immunotherapy may express a chimeric autoantibody receptor, such as a CAAR T cell. The pDCs are expected to enhance the activity of the ACT immunotherapy against the immune cells, such as B-cells or macrophages that express auto-reactive antibodies or otherwise cause inflammation and/or autoimmunity, and so be useful for treating autoimmune diseases and inflammatory diseases.

Suitable antigens that may be expressed by the ACT immunotherapy and bound by the pDC receptor include any appropriate autoantigen that mediates disease. For example, suitable antigens include autoantigens in multiple sclerosis, such as MBP, MOG, PLP, MAG, MOBP, CNPase, S100ß and Transaldolase; autoantigens in myasthenia gravis, such as nAChR, MuSK and LRP4; autoantigens in diabetes type 1 such as insulin, IA-2, GAD-65, ZnT8, IGRP and Chromogranin A; autoantigens in rheumatoid arthritis such as Fc-part of immunoglobulins citrullinated antigens, carbamylated antigens, 65-kDa heat-shock protein, cartilage glycoprotein-39 and aggrecan G1; autoantigens in neuromyelitis optica, such as AQP-4 and MOG; autoantigens in autoimmune encephalitis such as NMDA-receptor, AMPA-receptor, GABAA-receptor, GABAB-receptor, Gly-receptor, DPPX, GluR5, VGKC-complex and Hu.

In certain embodiments, the pDCs express at least one antigen that is bound by a receptor expressed by the ACT immunotherapy and the antigen is expressed on the surface of immune cells such as B-cells or macrophages that cause the autoimmune or inflammatory disease. The pDCs are expected to enhance the activity of the ACT immunotherapy against B-cells, thereby enhancing treatment of diseases including autoimmune diseases and inflammatory diseases, transplant rejection and GVHD.

Suitable antigens that may be expressed by the pDCs and bound by the ACT therapy targeting B-cells or macrophages include CD20 and CD19.

In the therapeutic methods of the invention, engineered cells are administered to a subject already suffering from an autoimmune or inflammatory disease, in an amount sufficient to cure, alleviate or reduce the frequency of one or more symptoms. An amount adequate to accomplish this is defined as “therapeutically effective amount”. The subject may have been identified as suffering from an autoimmune disease and being suitable for an ACT immunotherapy by any suitable means.

In a preferred embodiment, the pDCs administered according to the method are immunogenic. In some embodiments, the pDCs stimulate an immune response against the antigen. In some embodiments, treatment promotes the secretion of CXCL10 within the subject. In some embodiments, the pDCs administered according to the method stimulate the ACT to secrete CXCL10. In some embodiments, the pDCs and/or ACT secrete CXCL10.

### Methods for Treating Infectious Diseases

In preferred embodiments of the invention, the methods and cells of the invention are for use in treating an infectious disease. ACTs have been developed for treating infectious diseases (as reported in, for example, Seif et al. 2019, Front. Immunol. 10:2711. doi: 10.3389/fimmu.2019.02711 and Kumaresan, et al. Front Immunol. 2017; 8:1939). The ability of pDCs to improve the activity and proliferation of an ACT immunotherapy and to reduce exhaustion are expected to be particularly useful for treating infectious diseases.

In preferred embodiments, the infectious disease is a viral or fungal infection, such as infection of Influenza, Coronavirus, RSV, Measles, Parainfluenza, Zikavirus, Dengue virus, HIV, HBV, HCV, human cytomegalovirus (CMV), Epstein-Barr virus (EBV), Aspergillus spp., such as Aspergillus fumigatus, Candida spp., such as C. albicans, Mucorales, Cryptococcus, spp., such as Cryptococcus neoformans or Cryptococcus gattii, or Pneumocystis jirovecii. The methods of the invention are expected to be particularly useful for treating chronic infections.

In methods of treating infectious disease, the pDCs will generally express at least one antigen that is bound by a receptor expressed by the adoptive cell transfer immunotherapy and the at least one antigen will be a pathogen antigen. The ACT stimulated in accordance with the invention will then target the pathogen via the antigen and clear the infectious agent. In certain embodiments, the antigen is selected from: Influenza hemagglutinin antigens, Influenza neuraminidase antigens, coronavirus spike protein, RSV F protein, MeV N protein, Parainfluenza hemagglutinin antigens, Parainfluenza neuraminidase antigens, ZIKVE, NS1, NS3, NS4B, and NS5 proteins, Dengue virus C protein, M protein, E protein, and NS1 protein, gp120, gp41, Env, HBV surface antigen, HBV-surface proteins S and L, HCV E2 glycoprotein, CMV glycoprotein B, fungal beta glucan.

In the therapeutic methods of the invention, engineered cells are administered to a subject already suffering from an infectious disease, in an amount sufficient to cure, alleviate or reduce the frequency of one or more symptoms and/or in an amount sufficient to reduce infection load. An amount adequate to accomplish this is defined as “therapeutically effective amount”. The subject may have been identified as suffering from an infectious disease and being suitable for an ACT immunotherapy by any suitable means.

In a preferred embodiment, the pDCs administered according to the method are immunogenic. In some embodiments, the pDCs stimulate an immune response against the antigen. In some embodiments, treatment promotes the secretion of CXCL10 within the subject. In some embodiments, the pDCs administered according to the method stimulate the ACT to secrete CXCL10. In some embodiments, the pDCs and/or ACT secrete CXCL10.

### Methods for Pre-Conditioning an ACT Immunotherapy

The engineered pDCs of the invention may be used to pre-condition an ACT immunotherapy. The ACT immunotherapy may be co-cultured with the engineered pDCs prior to administration. Co-culturing the ACT immunotherapy with the engineered pDCs of the invention may comprise incubating the ACT immunotherapy with the engineered pDCs. The cells may be incubated for 24 hours. The cells may be incubated for more than 24 hours. Co-culturing may be performed during expansion of the ACT immunotherapy. Pre-conditioning the ACT immunotherapy with the engineered pDCs of the invention may improve certain characteristics of the ACT immunotherapy and thus make the treatment more effective. For example, pre-conditioning may result in improved proliferation of the ACT immunotherapy. Pre-conditioning may also increase the levels of cytokines produced by the ACT immunotherapy. Pre-conditioning may result in an ACT immunotherapy with enhanced ability to kill target cells and with better protection against exhaustion.

The ability of a pDC expressing at least one antigen which is bound by a receptor expressed by an ACT immunotherapy to pre-condition said ACT immunotherapy may be tested by challenging the pre-conditioned ACT immunotherapy with tumour cells and measuring the number of live tumour cells after a given incubation period. This can be compared to the number of live tumour cells produced by a corresponding method using pDCs not expressing the at least one antigen. A pre-conditioned ACT immunotherapy has an improved ability to kill tumour cells if the number of live tumour cells after incubation with the pre-conditioned ACT immunotherapy is less than the number of live tumour cells after incubation with a corresponding ACT immunotherapy which has not been pre-conditioned. One such suitable assay is set out in Example 6.

In some embodiments, the invention provides a method of treating a disease comprising administering an ACT immunotherapy, wherein the method comprises a step of pre-conditioning the ACT immunotherapy with pDCs expressing at least one antigen that is bound by a receptor expressed by the ACT immunotherapy.

In some embodiments, the step of pre-conditioning comprises co-culturing the ACT immunotherapy with the pDCs. In some embodiments, the ratio of ACT immunotherapy cells to pDCs is between 2:1 and 1:2, such as between 1.5:1 and 1:1.5, or between 1.2:1 and 1:1.2, or preferably about 1:1. In some embodiments, the ratio of ACT immunotherapy cells to pDCs is between 1:0.4 and 1:01, such as between 1:0.3 and 1:0.1, or preferably about 1:0.2. In some embodiments, the ratio of ACT immunotherapy cells to pDCs is between 1:2 and 1:0.05, such as between 1:1.5 and 1:0.1, or between 1:1.1 and 1:0.15, or between 1:1 and 1:0.2. For example, the ratio may be about 1:2, 1:1.9, 1:1.8, 1:1.7, 1:1.6, 1:1.5, 1:1.4, 1:1.3, 1:1.2, 1:1.1, 1:1, 1:0.9, 1:0.8, 1:0.7, 1:0.6, 1:0.5, 1:0.4, 1:0.3, 1:0.2, 1:0.1, between 1:2 and 1:0.2, between 1:1 and 1:0.2, or around 1:1. In some embodiments, the ratio of ACT immunotherapy cells to pDCs is 1:1. In some embodiments, the ratio of ACT immunotherapy cells to pDCs is 1:0.2.

In some embodiments, the invention provides a method of pre-conditioning an ACT immunotherapy comprising co-culturing the ACT immunotherapy with pDCs expressing at least one antigen that is bound by a receptor expressed by the ACT immunotherapy.

In some embodiments, pre-conditioning the ACT immunotherapy increases the level of cytokines produced by the ACT immunotherapy. In some embodiments, the cytokines comprise IL-2, TFNα, and IFNγ.

In some embodiments, the pre-conditioning increases the levels of cytokines produced by the ACT immunotherapy when compared to a corresponding method comprising the step of co-culturing the ACT immunotherapy with pDCs not expressing at least one antigen that is bound by a receptor expressed by the ACT immunotherapy.

In some embodiments, the pre-conditioning increases the levels of cytokines produced by the ACT immunotherapy when compared to a corresponding method comprising the step of co-culturing the ACT immunotherapy with tumour cells expressing at least one antigen that is bound by a receptor expressed by the ACT immunotherapy.

In some embodiments, pre-conditioning the ACT immunotherapy increases the proliferation of the ACT immunotherapy.

In some embodiments, the pre-conditioning increases the proliferation of the ACT immunotherapy when compared to a corresponding method comprising the step of co-culturing the ACT immunotherapy with pDCs not expressing at least one antigen that is bound by a receptor expressed by the ACT immunotherapy.

In some embodiments, the pre-conditioning increases the proliferation of the ACT immunotherapy when compared to a corresponding method comprising the step of co-culturing the ACT immunotherapy with tumour cells expressing at least one antigen that is bound by a receptor expressed by the ACT immunotherapy.

In some embodiments, pre-conditioning the ACT immunotherapy decreases the level of exhaustion, such as measured by expression of exhaustion markers, of the ACT immunotherapy.

In some embodiments, the pre-conditioning decreases the level of exhaustion, such as measured by expression of exhaustion markers, of the ACT immunotherapy when compared to a corresponding method comprising the step of co-culturing the ACT immunotherapy with pDCs not expressing at least one antigen that is bound by a receptor expressed by the ACT immunotherapy.

In some embodiments, the pre-conditioning decreases the level of exhaustion, such as measured by expression of exhaustion markers, of the ACT immunotherapy when compared to a corresponding method comprising the step of co-culturing the ACT immunotherapy with tumour cells expressing at least one antigen that is bound by a receptor expressed by the ACT immunotherapy.

Any suitable known exhaustion markers may be used (for example as reported in Wherry and Kuracki, 2015, Nat Rev Immunol. 2015 August; 15(8): 486-499 and/or Tang et al. Biomed Res Int. 2021; 2021:6616391. ). Markers characteristic of exhausted T cells, which may be reduced upon co-culturing or co-administration with pDCs according to the invention include inhibitory markers, PD1, CD44, T-bet, EOMES, CD244, CD160 and Blimp-1. Markers characteristics of active and effective T cells, which may be increased upon co-culturing or co-administration with pDCs according to the invention include CD28, CD44, LY6C, CD57, NK cell receptor markers, and killer cell lectin-like receptor subfamily G member 1 (KLRG1).

In a preferred embodiment, the pDCs administered according to the method are immunogenic. In some embodiments, the pDCs stimulate an immune response against the antigen. In some embodiments, treatment promotes the secretion of CXCL10 within the subject. In some embodiments, the pDCs administered according to the method stimulate the ACT to secrete CXCL10. In some embodiments, the pDCs and/or ACT secrete CXCL10.

### Methods for Generating Cells of the Invention

The engineered pDCs may be generated by any appropriate method. Exemplary methods for generating pDCs in significant amounts are provided in WO2018/206577. The invention also provides methods of generating engineered plasmacytoid dendritic cells.

In certain embodiments, the method for producing an engineered plasmacytoid dendritic cell (pDCs) comprises:
providing hematopoietic stem progenitor cells (HSPCs)
transfecting/transducing said HSPCs with a vector comprising an expression cassette comprising a transgene encoding at least one antigen that is bound by a receptor expressed by an ACT immunotherapy, or encoding at least one receptor that binds an antigen expressed an ACT immunotherapy,
differentiating the HSPCs into pDCs.

In certain embodiments, the method for producing an engineered plasmacytoid dendritic cell (pDCs) comprises:
providing hematopoietic stem progenitor cells (HSPCs)
differentiating the HSPCs into pDCs,
transfecting/transducing said pDCs with a vector comprising an expression cassette comprising a transgene encoding at least one antigen that is bound by a receptor expressed by an ACT immunotherapy, or encoding at least one receptor that binds an antigen expressed an ACT immunotherapy.

In certain embodiments, differentiating the HSPCs into pDCs may comprise incubating said HSPCs in one or more media, which media may typically comprise one or more cytokines, growth factors, interferons (IFNs) and/or aryl hydrocarbon receptor (AHR) antagonists (such as stemregenin-1), whereby said HSPCs are differentiated into precursor-pDCs and into pDCs.

Accordingly, in certain embodiments, the method for producing an engineered plasmacytoid dendritic cell (pDCs) comprises:
providing hematopoietic stem progenitor cells (HSPCs),
incubating said HSPCs in one or more media, which media may typically comprise one or more cytokines, growth factors, interferons (IFNs) and/or aryl hydrocarbon receptor (AHR) antagonists (such as stemregenin-1), whereby said HSPCs are differentiated into precursor-pDCs and into pDCs, and
transfecting/transducing said HSPCs prior to differentiation, or transfecting/transducing said HSPCs subsequent to differentiation, with a vector comprising an expression cassette comprising a transgene encoding at least one antigen that is bound by a receptor expressed by an ACT immunotherapy, or encoding at least one receptor that binds an antigen expressed an ACT immunotherapy.

Transfecting/transducing the cells can be achieved by any appropriate technique.

The vector may be an appropriate vector, such as selected from the group consisting of a viral construct, an mRNA, a plasmid or a cosmid.

In some embodiments the vector is a viral construct. In some embodiments, the viral construct is an AAV construct, an adenoviral construct, a lentiviral construct, or a retroviral construct. The construct may comprise a reporter gene such as GFP, mCherry, truncated EGFR, or truncated tNGFR, or the extracellular domain of the CAR or synNotch may contain an epitope, to aid sorting of pDCs with the construct. In some embodiments, the viral construct is a lentiviral construct and transduction is performed using retronectin-coated plates or using lentiboost and protamine sulfate. In preferred embodiments of the invention, the method includes the step of transducing the HSPCs with a viral construct before the step of differentiating the HSPCs into pDCs. pDCs differentiated from HSPCs transduced with a viral construct encoding an antigen may stably express the antigen.

In some embodiments the vector is an mRNA encoding an antigen. The mRNA may be delivered to the cell using any appropriate technique, such as electroporation or using a lipid nanoparticle (LNP). In a preferred embodiment, the step of transfecting the pDCs with an LNP comprising an mRNA encoding an antigen occurs after the step of differentiating the HSPCs into pDCs. pDCs transfected with an LNP comprising an mRNA encoding an antigen may transiently express the antigen.

In certain embodiments, the heterologous nucleic acid is introduced using a site-specific DNA editing such as TALEN, zinc finger or CRISPR/Cas.

In preferred embodiments, CD34⁺ HSPC are transduced or transfected and then differentiated into pDCs.

In certain embodiments, the method for producing an engineered plasmacytoid dendritic cell (pDCs) comprises:
providing hematopoietic stem progenitor cells (HSPCs)
transfecting/transducing said HSPCs with a vector comprising an expression cassette comprising a transgene encoding the at least one antigen or receptor,
incubating said HSPCs in a first medium comprising cytokines and growth factor whereby said HSPCs are differentiated into precursor-pDCs
adding interferons (IFNs) to said first medium to obtain a second medium whereby said precursor-pDCs are differentiated into pDCs

In certain embodiments, the method for producing an engineered plasmacytoid dendritic cell (pDCs) comprises:
providing hematopoietic stem progenitor cells (HSPCs)
transfecting/transducing said HSPCs with a vector comprising an expression cassette comprising a transgene encoding the at least one antigen or receptor,
incubating said HSPCs in a first medium comprising cytokines and growth factor whereby said HSPCs are differentiated into precursor-pDCs
adding stem cell factor (SCF) and StemRegnin 1 (SR1) in a first medium to obtain high yield of pre-cursor pDCs
providing a second medium comprising interferons (IFNs)
adding interferons (IFNs) to said a second medium to said first medium comprising pre-cursor pDCs, whereby said precursor-pDCs are transformed into to obtain a high yield of fully activated and differentiated pDCs a second medium whereby said precursor-pDCs are differentiated into pDCs.

In some embodiments, the invention uses pDCs extracted from blood or bone marrow. Any appropriate technique may be used for isolating pDCs from blood or bone marrow. Such cells may be transfected with a vector comprising an expression cassette comprising a transgene encoding the at least one antigen or receptor using any appropriate method. In some embodiments, the invention uses pDCs derived from induced pluripotent stem cells (iPSCs) or embryonic stem cells (ESCs). Any appropriate technique may be used for differentiating iPSCs and ESCs into HSPCs and into pDCs. Exemplary protocols are provided herein for differentiating HSPCs into pDCs and the production of pDCs from ESCs and iPSCs are disclosed in, for example, Li et al. World J Stem Cells. 2014 Jan. 26; 6(1): 1-10.

In some embodiments of the invention, the pDCs comprise a heterologous nucleic acid encoding the at least one antigen or receptor. In some embodiments, the heterologous nucleic acid is integrated into the genome of the engineered cell. In some embodiments, the heterologous nucleic acid is not integrated into the genome of the engineered cell. In some embodiments, the heterologous nucleic acid is introduced by a transposase, retrotransposase, episomal plasmid, mRNA, or random integration. In certain embodiments, the heterologous nucleic acid is introduced with a gene editing system such as TALEN, zinc finger or CRISPR/Cas9.

In preferred embodiments said second medium comprises IFN-γ and/or IFN-β. In another embodiment said second medium further comprises IL-3. Preferably, said second medium comprises IL-3, IFN-γ and IFN-β.

The precursor-pDCs may for example be incubated for at least 24 hours in said second medium. Said precursor-pDCs, may incubated for 24 to 72 hours in said second medium. Preferably, said precursor pDCs are incubated for around 24 hours, such as 20-28, 22-26 or 24 hours.

In one embodiment said first medium comprises Flt3 ligand, thrombopoietin and/or interleukin-3. In another embodiment said first medium further comprises stem cell factor and StemRegenin 1. In another embodiment said first medium further comprises stem cell factor and UM 171. In another embodiment said first medium further comprises RPMI medium supplemented with fetal calf serum (FCS). In another embodiment said first medium comprises serum-free medium (SFEM or GMP DC Medium). Preferably, said first medium comprises Flt3 ligand, thrombopoietin, SCF, interleukin-3 and StemRegenin 1.

The HSPCs may for example be incubated for 21 days in said first medium.

In one embodiment the method as described herein, further comprises a step of immunomagnetic negative selection to enrich for differentiated pDCs.

“Hematopoietic stem cells” (HSCs) as used herein are multipotent stem cells that are capable of giving rise to all blood cell types including myeloid lineages and lymphoid lineages. Myeloid lineages may for example include monocytes and macrophages, neutrophils, basophils, eosinophils, erythrocytes, megakaryocytes/platelets and dendritic cells, whereas lymphoid lineages may include T-cells, B-cells and NK-cells.

In a preferred embodiment HSCs are Hematopoietic stem and progenitor cells (HSPCs). HSCs or HSPCs are found in the bone marrow of humans, such as in the pelvis, femur, and sternum. They are also found in umbilical cord blood and in peripheral blood.

Stem and progenitor cells can be taken from the pelvis, at the iliac crest, using a needle and syringe. The cells can be removed as liquid for example to perform a smear to look at the cell morphology or they can be removed via a core biopsy for example to maintain the architecture or relationship of the cells to each other and to the bone.

The HSCs or HSPCs may also be harvested from peripheral blood. To harvest HSCs or HSPCs from the circulating peripheral blood, blood donors can be injected with a cytokine that induces cells to leave the bone marrow and circulate in the blood vessels. The cytokine may for example be selected from the group consisting of granulocyte-colony stimulating factor (G-CSF), Plerixafor, GM-CSF granulocyte-macrophage colony-stimulating factor (GM-CSF) and cyclophosphamide. They are usually given as an injection into the fatty tissue under the skin every day for about 4-6 days.

The HSCs or HSPCs may also be harvested or purified from bone marrow. Stem cells are 10-100 times more concentrated in bone marrow than in peripheral blood. The hip (pelvic) bone contains the largest amount of active marrow in the body and large numbers of stem cells. Harvesting stem cells from the bone marrow is usually done in the operating room.

HSCs or HSPCs may also be purified from human umbilical cord blood (UCB). In this method, blood is collected from the umbilical cord shortly after a baby is born. The volume of stem cells collected per donation is quite small, so these cells are usually used for children or small adults.

The first medium is a differentiation medium, wherein HSCs are differentiated into precursor-pDCs. Thus, the first medium comprises differentiation factors.

Before differentiation of HSCs into precursor-pDCs, the HSCs may be cultured in a culture medium not comprising differentiation factors. The culture medium may be supplemented with conventional cell culture components such as serum, such as for example fetal calf serum, b-mercaptoethanol, antibiotics, such as penicillin and/or streptomycin, nutrients, and/or nonessential amino acids. Conventional cell culture components can also be substituted for conventional serum-free medium supplemented with conventional penicillin and/or streptomycin.

To initiate differentiation of HSCs into precursor-pDCs, differentiation factors, such as Flt3 ligand, thrombopoietin and/or at least one interleukin selected from interleukin-3, IFN-b and PGE2 are added to the medium. SCF and/or SR1 can also be used.

Thus, in a preferred embodiment said first medium comprises Flt3 ligand, thrombopoietin and/or at least one interleukin selected from interleukin-3, IFN-b and PGE2. More preferably, said first medium comprises Flt3 ligand, thrombopoietin and/or interleukin-3. In another preferred embodiment, the first medium comprises SCF and/or SR1.

Appropriate culture media can be prepared by the skilled person, for example using the guidance in WO 2018/206577.

The HSPCs are incubated in the first medium under conditions that are typical for human cell cultures and well known to the skilled person. Typical conditions for incubation of cell cultures are for example a temperature of 37° C., 95% humidity and 5% CO₂.

In one embodiment the HSPCs are incubated for at least 1 day, such as at least 2 days, at least 3 days, such as for example at least 4 days, such as at least 5 days, at least 6 days, such as for example at least 7 days, such as at least 8 days, at least 9 days, such as for example at least 10 days, such as at least 12 days, at least 14 days in said first medium. In a more preferred embodiment the culture is incubated for at least 16 days, such as at least 18 days, at least 20 days or such as for example at least 21 days in said first medium.

The HSCs may for example be incubated for 1 week, 2 weeks, 3 weeks or 4 weeks in said first medium. In a preferred embodiment said HSPCs are incubated for 21 days in said first medium.

In one embodiment the first medium is refreshed during the incubation period. The medium may for example be refreshed every second day, every third day or every fourth day during the incubation period. The first medium is preferably refreshed with medium containing one or more components of the first medium as described herein and above. Preferably the medium is refreshed with medium comprising the cytokines.

After incubation of HSPCs in the first medium, wherein HSCs are differentiated into precursor-pDCs, IFNs are added to the first medium thereby obtaining a second medium.

Alternatively, a second medium is provided, which comprises IFNs, such as IFN type I, IFN type II and/or IFN type III.

In one embodiment said second medium comprises IFN-«, IFN-γ and/or IFN-β.

In one preferred embodiment said second medium comprises IFN-γ and/or IFN-β. Preferably, said second medium comprises IFN-γ and IFN-β.

In another preferred embodiment said second medium comprises interleukin-3 (IL-3). In the embodiment, wherein the first medium comprises IL-3, IL-3 may be added to the medium again, for example together with the interferons. It is understood that the three components can be added in any order. In a particular preferred embodiment said second medium comprises IFN-γ, IFN-β and IL-3.

The precursor-pDCs are incubated in the second medium under conditions that are typical for human cell cultures and well known to the skilled person. Typical conditions for incubation of cell cultures are for example a temperature of 37°° C., 95% humidity and 5% CO₂.

In one embodiment said precursor-pDCs are incubated in said second medium for at least 1 hour, such as at least 5 hours, such as for example at least 10 hours, such as at least 15 hours or such as at least 20 hours in said second medium. In one preferred embodiment precursor-pDCs are incubated for at least 24 hours in said second medium.

In another embodiment said precursor-pDCs are incubated in said second medium for at least 1 day, at least two days, at least three days or at least 4 days.

In some embodiments, the methods of the invention include a step of priming the pDCs before administration and/or the step of pre-conditioning. In some embodiments, the step of priming the pDCs comprises incubating the pDCs with type I IFN and/or type II IFN. The pDCs may be incubated with the type I IFN and/or type II IFN for at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours. In a preferred embodiment, the pDCs are incubated with the type I IFN and/or type II IFN for 24 hours. The step of priming the pDCs may increase the level of IFNα expressed by the pDCs. This may improve the ability of the pDCs to activate and/or pre-condition an ACT immunotherapy.

### General

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polypeptide” includes “polypeptides”, and the like.

Unless specifically prohibited, the steps of a method disclosed herein may be performed in any appropriate order and the order in which the steps are listed should not be considered limiting.

All publications, patents and patent applications cited herein are hereby incorporated by reference in their entirety.

### Example 1

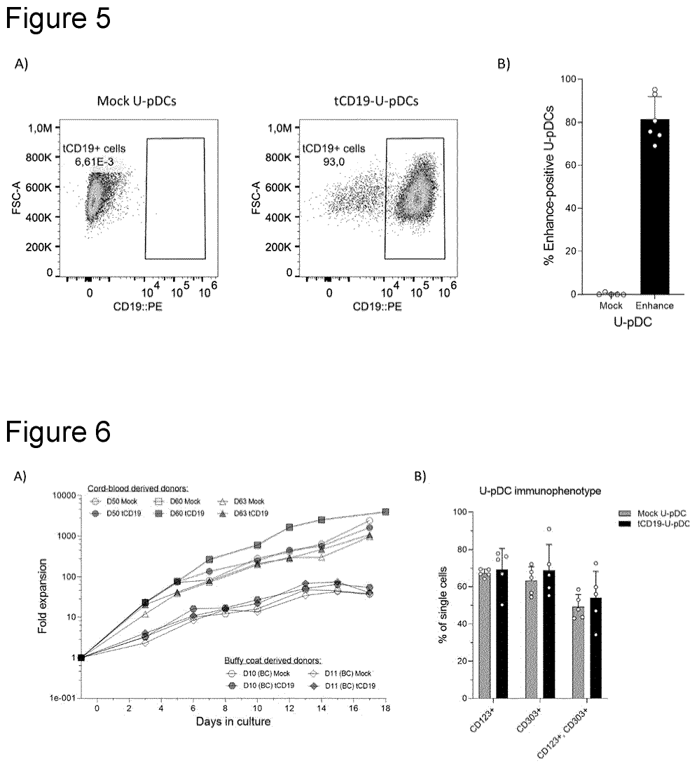

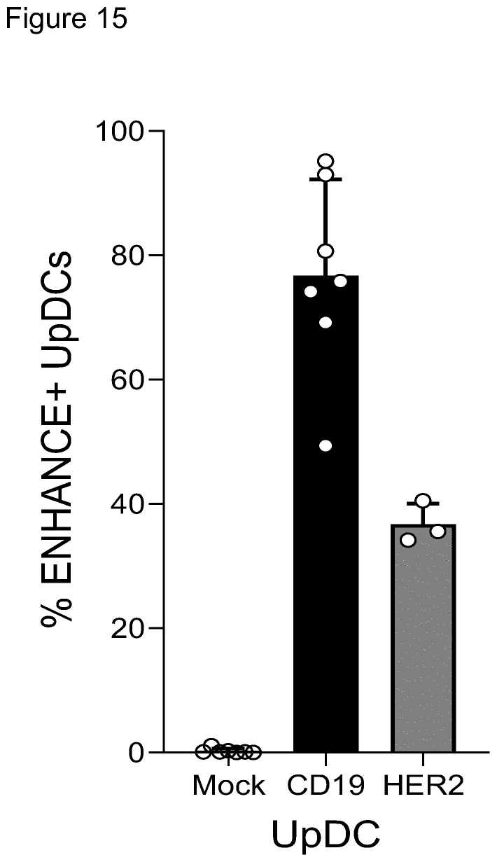

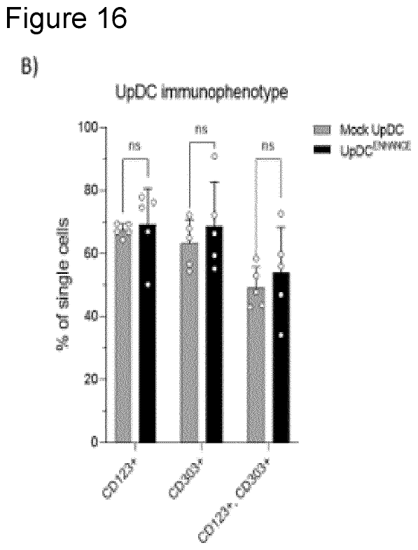

Hematopoietic stem and progenitor cells (HSPCs) were transduced with a lentiviral vector encoding intracellularly truncated CD19 (FIG. 2). The cells were subsequently successfully differentiated into U-pDCs. FIG. 5A shows representative flow cytometry plots of the expression of the truncated CD19 (tCD19) construct in U-pDCs^{ENHANCE} compared with Mock U-pDCs (untransduced). This approach was repeated in six individual donors to demonstrate the robustness of the method (see FIG. 5B). Seven further donors were used to generate both tCD19-and tHER2-UpDCs^{ENHANCE} in FIG. 15. The examples clearly demonstrate that U-pDCs can be genetically modified to stably express truncated CAR antigens.

### Example 2

Untransduced (Mock) or lentivirally transduced (tCD19) CD34⁺ cells were differentiated into U-pDCs, and subsequently IFN-primed for 24 hours and analyzed for pDC-marker expression by flow cytometry. FIG. 6A shows similar cell expansion of Mock versus tCD19-transduced conditions during differentiation from CD34⁺ cells to U-pDCs for multiple individual cord blood-and buffy coat donors. These donors were consolidated in FIG. 6B and FIG. showing similar frequency of pDC-marker expression (CD123 and CD303) on IFN-primed Mock-and tCD19-U-pDCs^{ENHANCE} The example confirms that U-pDCs can be genetically modified to stably express truncated CAR-antigens without major impact on the cell expansion during differentiation or pDC immunophenotyping.

### Example 3

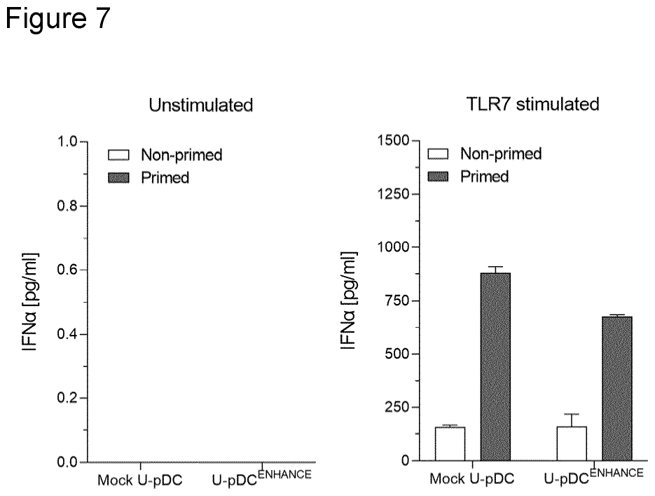

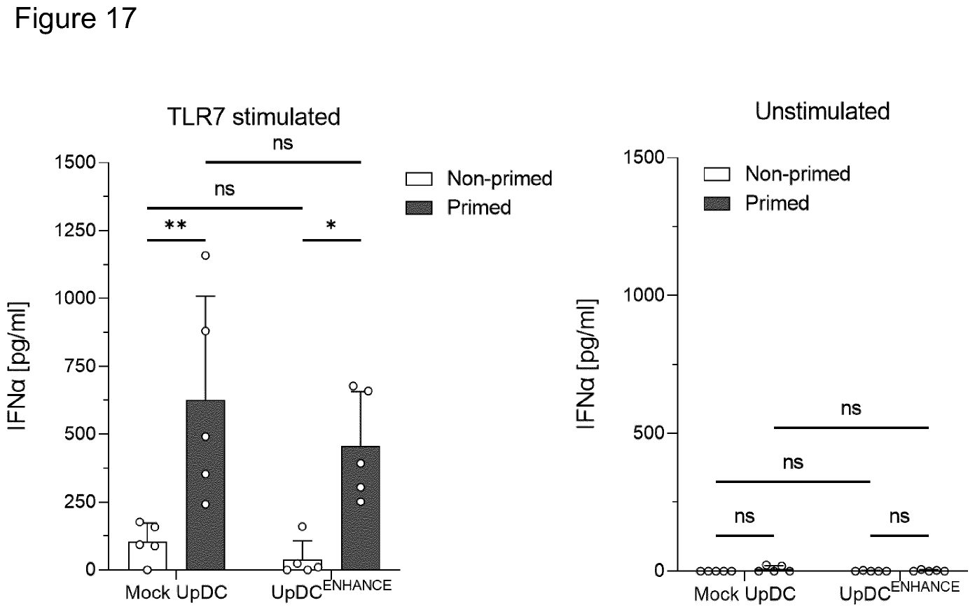

Primed or non-primed U-pDCs, where 95% of the cells express a truncated CD19 (U-pDC^{ENHANCE}), or U-pDCs not expressing the construct (Mock U-pDC), were stimulated for 20 hours with agonists directed against TLR7 (R837) or remained unstimulated. Supernatants were subsequently harvested, and IFNα-responses were quantified using commercially available IFNα ELISA. FIG. 7 illustrates that pDC functionality measured by the type I IFN response (one of the hallmarks of pDC activation) is not affected in a population of U-pDCs expressing the intracellularly truncated CAR antigen. This experiment was repeated using U-pDCs where 71-95% of the cells express a truncated CD19, and similar results were achieved (illustrated in FIG. 17). The example demonstrates that Mock UpDCs and UpDC^{ENHANCE} have a comparable ability to respond to a TLR7 agonist.

### Example 4

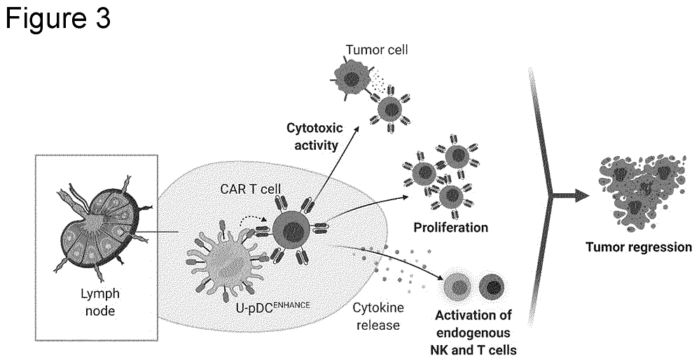

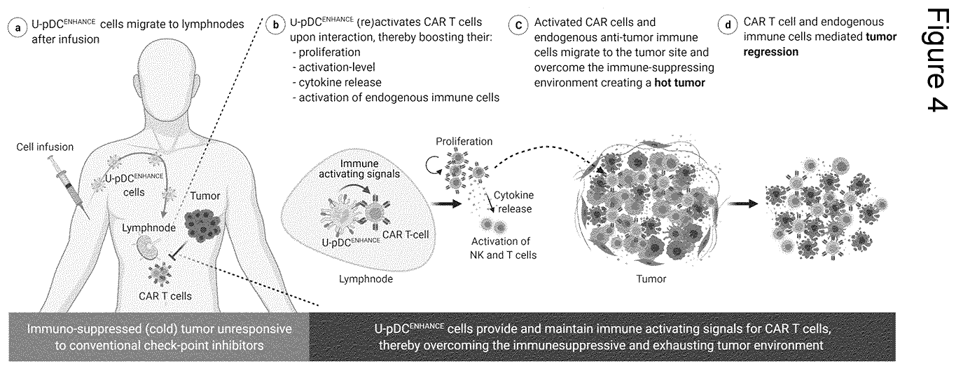

Chimeric antigen receptor (CAR) modified T cells express a synthetic construct that enables binding of a specific antigen on target cells and the subsequent CAR T cell activation. However, CAR T cells are prone to exhaustion due to the immune-suppressing microenvironment associated with solid tumors (FIG. 3). To overcome this, UpDCsENHANCE facilitates delivery of potent immune activating signals to the CAR T cells through the co-stimulatory molecules naturally expressed on UpDCs. This leads to strong activation of the CAR T cells resulting in increased proliferation, cytotoxic activity, cytokine production (e.g., IFNγ, IL-2, and TNFα), and decreased exhaustion (FIG. 3).

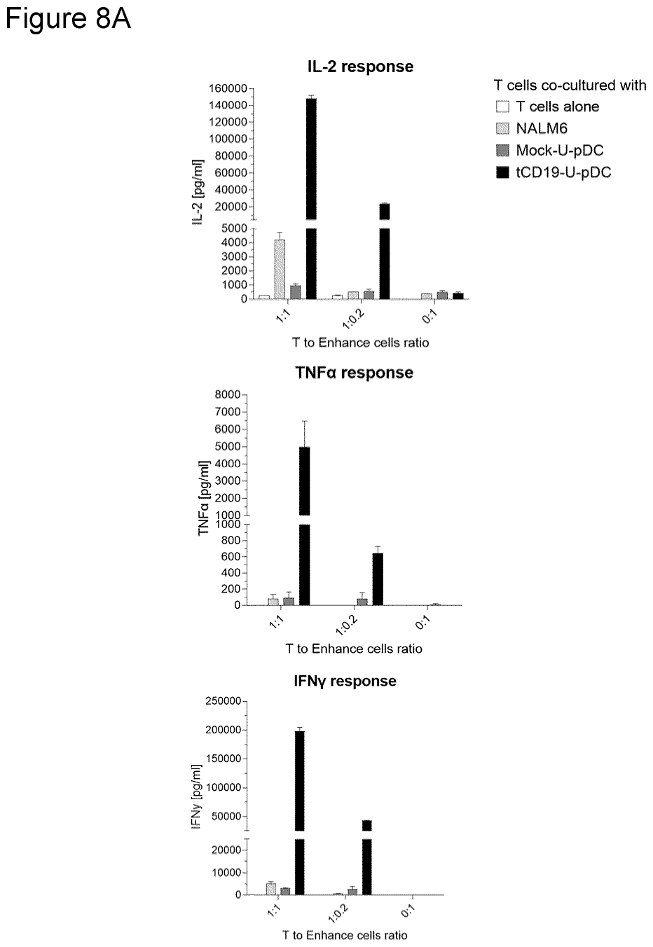

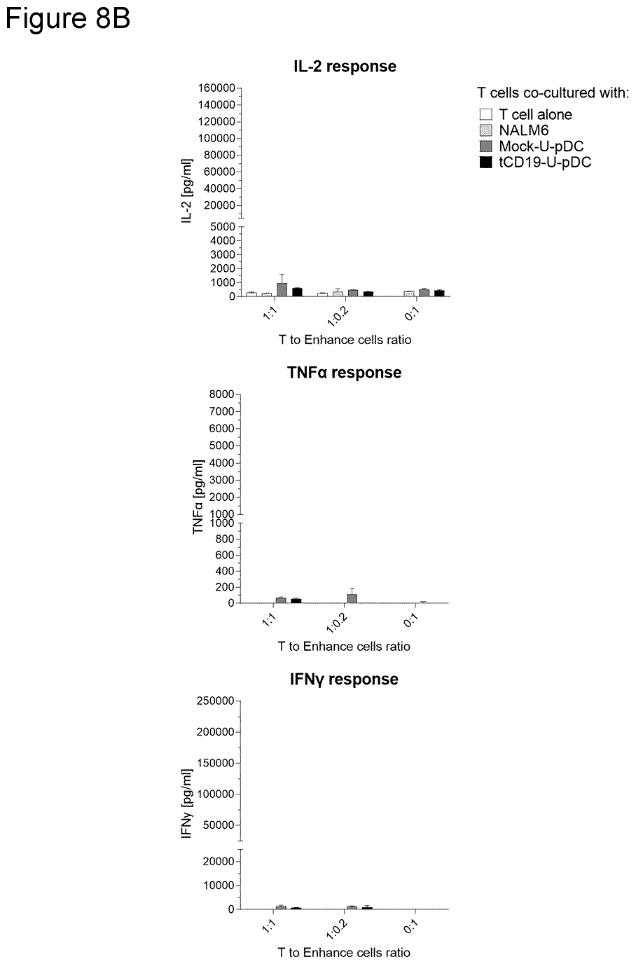

Primed tCD19⁺ U-pDCs^{ENHANCE} and Mock U-pDCs were co-cultured with anti-CD19 CAR T cells (FIG. 8A) or Mock T cells (FIG. 8B) at a T cell: Enhance cell ratio of 1:1 or 1:0.2 to assay the specific enhancing effect on CAR T cell activation. CAR-T cells co-cultured with CD19+ target tumour cells (NALM6) were included as activation control. Enhance U-pDCs and controls were additionally seeded without CAR T cells (0:1) to exclude potential background. After 24 hours of co-cultures, all supernatants were harvested and the levels of IL-2, TFNα, and IFNγ cytokines (hallmarks for T cell activation) were quantified by ELISA.

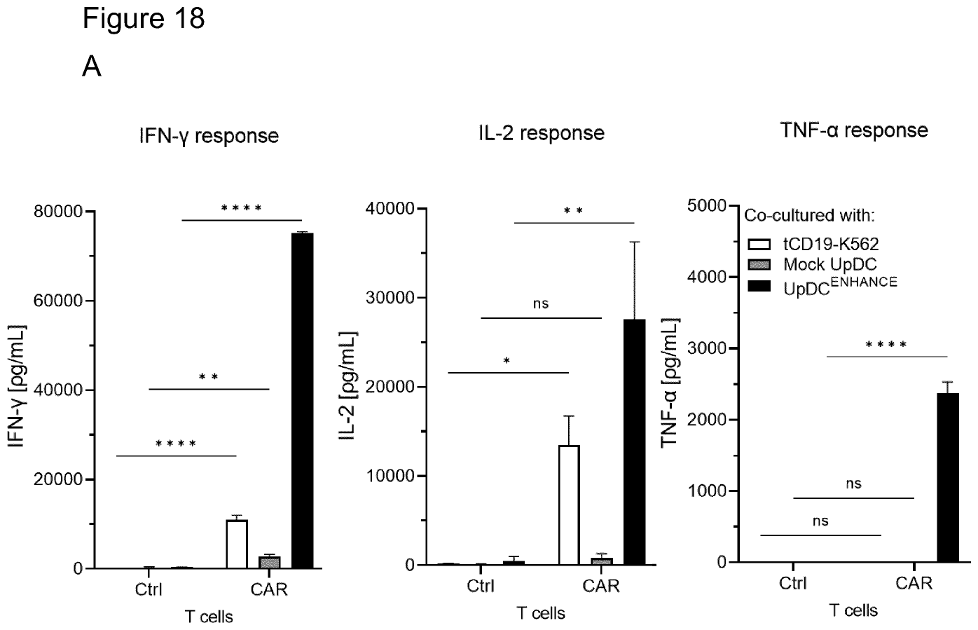

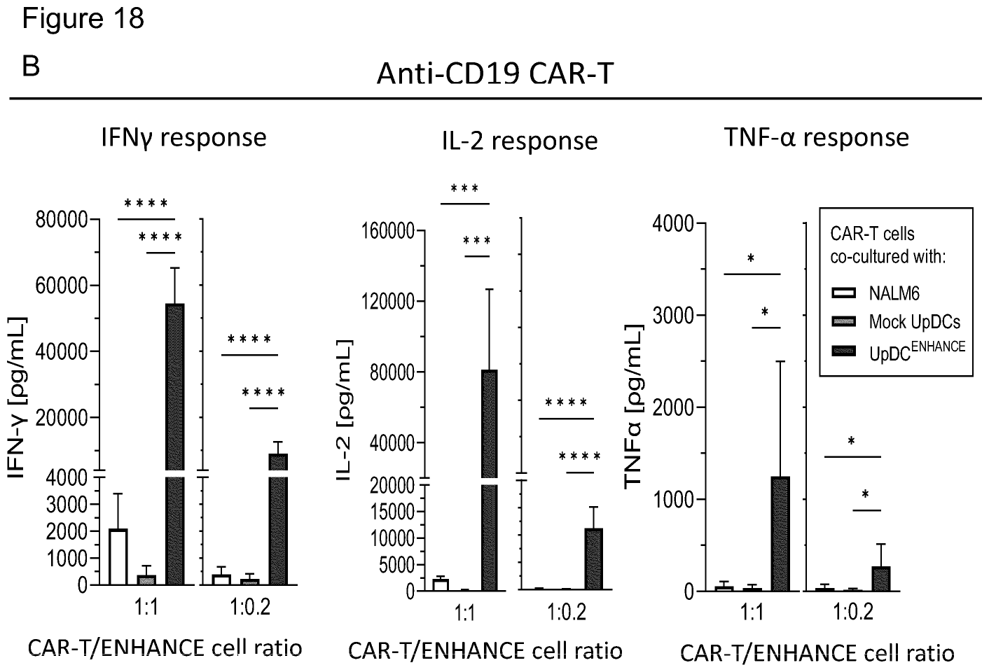

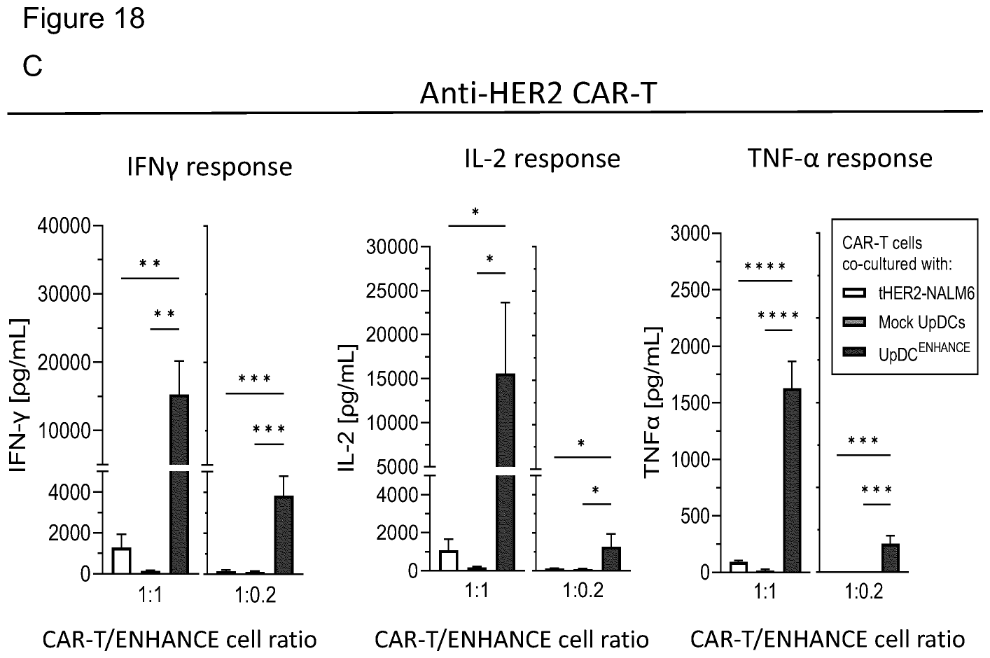

FIG. 8A demonstrate that anti-CD19 CAR T cells in co-culture with CAR-antigen (tCD19)-expressing U-pDC^{ENHANCE} cells specifically induce very high production of T cell activation-associated cytokines (IL-2, TFNα, and IFNγ) compared with Mock U-pDC and NALM6 co-cultures. The antigen specificity is supported by the data presented in FIG. 8B, showing minimal production of T cell activation-associated cytokines from Mock T cells across all co-culture conditions. This protocol was modified to produce the data of FIG. 18.

Primed tCD19⁺ UpDCs^{ENHANCE} and Mock UpDCs were co-cultured with anti-CD19 CAR T cells or control T cells (FIG. 18A) at a T cell: ENHANCE cell ratio of 1:1 to assay the specific enhancing effect on CAR T cell activation. CAR T cells co-cultured with the K562 target tumor cells overexpressing tCD19 antigen were included as activation control. After 48 hours of co-culture, all supernatants were harvested and the levels of IL-2, TFNα, and IFNγ cytokines (hallmarks for T cell activation) were quantified by commercially available ELISA. FIG. 8A demonstrates that anti-CD19 CAR T cells in co-culture with UpDC^{ENHANCE} specifically induces high production of T cell activation-associated cytokines (IL-2, TFNα, and IFNγ) compared with Mock UpDC and tCD19-K562 co-cultures. Emphasizing the specificity of the CAR T cell activation boost, only minimal effect is observed in control T cell co-cultures.

Further supporting the robustness of UpDCs^{ENHANCE}, both anti-CD19 CAR T (FIG. 18B) and anti-HER2 CAR T cells (FIG. 18C) were strongly boosted by co-culture with their corresponding UpDCs^{ENHANCE} compared with Mock UpDC and target tumor co-cultures. Furthermore, UpDCs^{ENHANCE} greatly boost CAR T activation at lower T cell: ENHANCE cell ratio (1:0.2), while the target tumor cell co-cultures show minimal activation (FIGS. 18B-C).

This Example demonstrates that CAR-antigen expressing U-pDCs potently and specifically activate the CAR T cells.

### Example 5

Anti-CD19-CAR T cells were stained with CellTrace Yellow dye and co-cultured with Mock U-pDCs or tCD19-U-pDCs^{ENHANCE} at a T cell: Enhance cell ratio of 1:1 or 1:0.2. CAR-T cells co-cultured with CD19-expressing target tumour cells (NALM6) were included as positive control. After 48 hours, the frequency of T cells that have gone through cell division was analyzed using flow cytometry. T cells cultured alone (T cells alone) were included as a control for background proliferation.

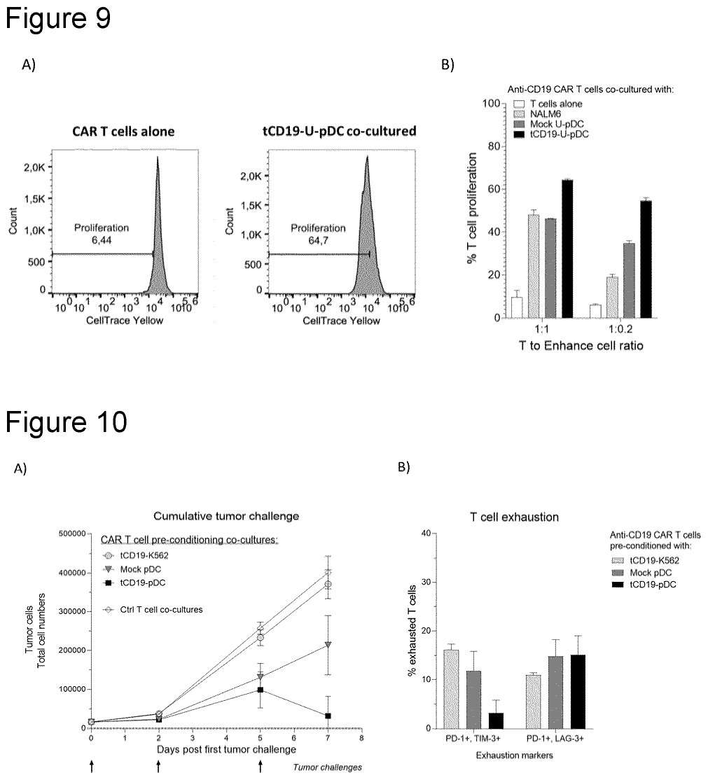

FIG. 9A shows representative flow cytometry plots of CAR T cell proliferation after 48-hour co-culture stimulation compared to unstimulated CAR T cells. Proliferation is detected as a loss in dye intensity (halved at each cell division). FIG. 9B shows a higher proportion of proliferating anti-CD19 CAR T cells when co-cultured with tCD19-U-pDC^{ENHANCE} cells compared with the control co-culture conditions (including stimulation by target tumour cells).

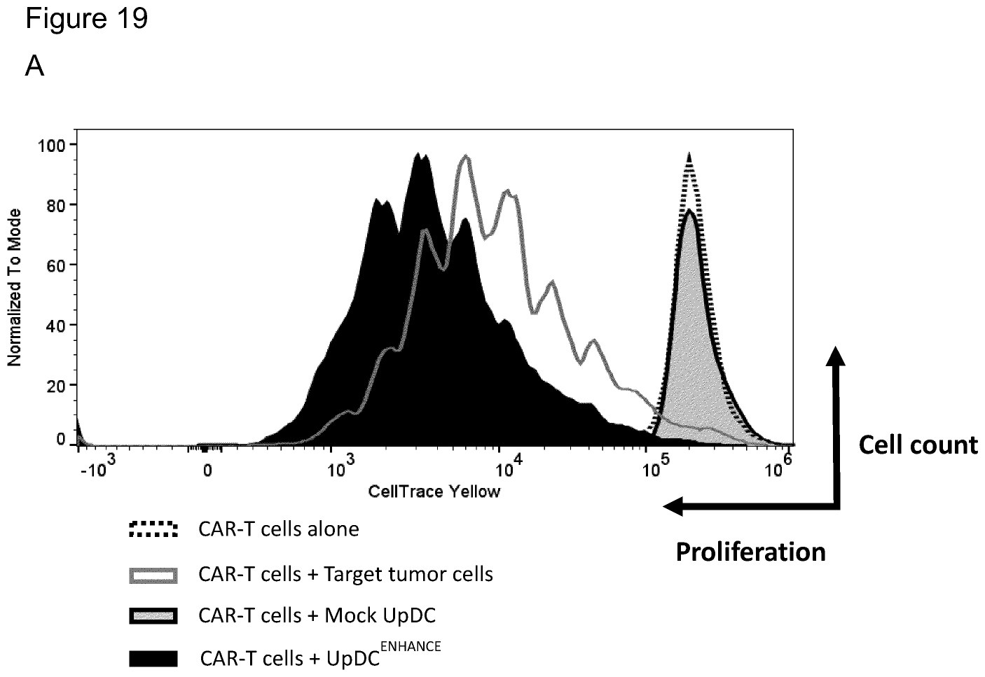

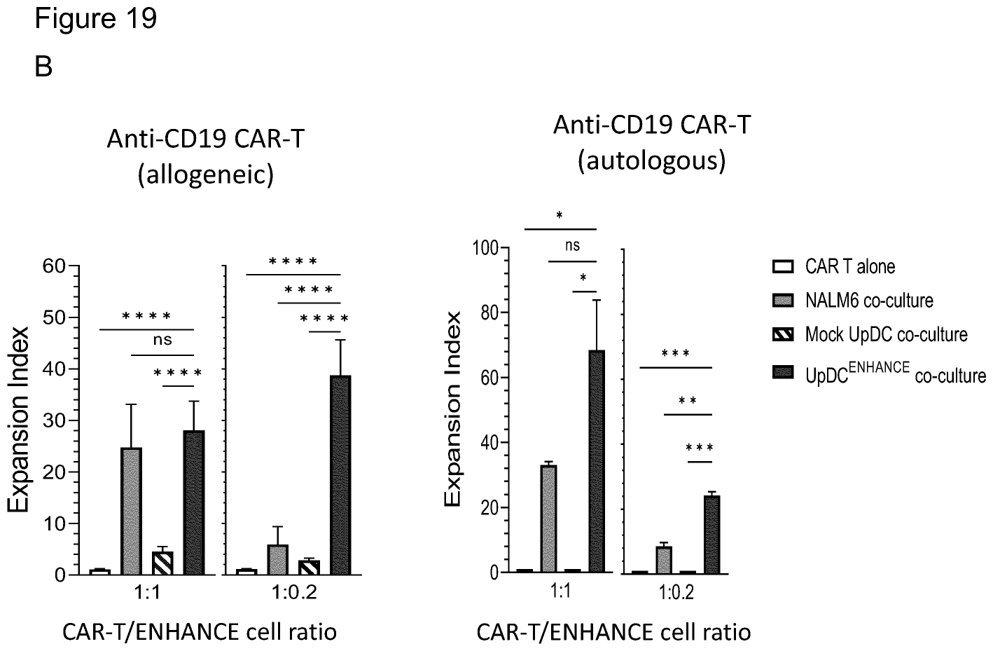

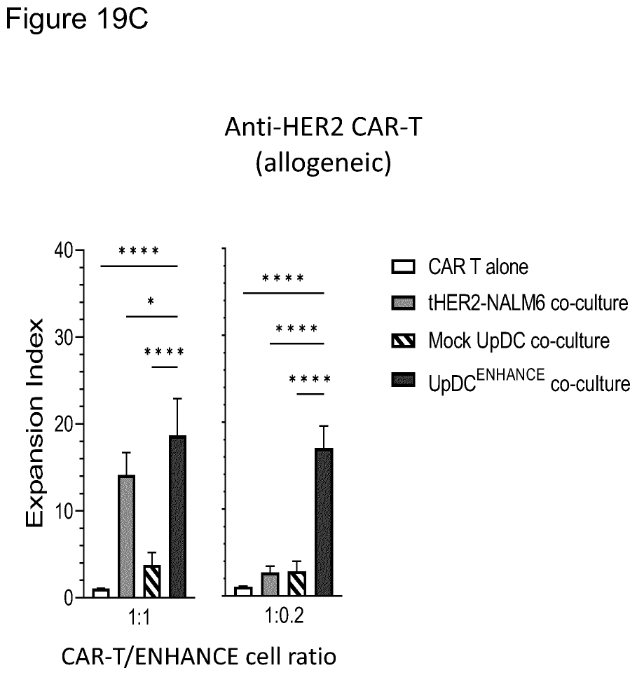

A modified protocol was used to generate the data of FIG. 19B. After 120 hours, the cells were harvested and analyzed for proliferation by flow cytometry. FIG. 19A shows representative flow cytometry plots of CAR T cell proliferation after 120-hour co-culture stimulation compared to unstimulated CAR T cells. CAR T cells when co-cultured with tCD19-U-pDC^{ENHANCE} cells display a markedly lower CellTrace intensity and therefore higher proliferation compared with the control co-culture conditions

FIG. 19B demonstrates that anti-CD19 CAR T cells co-cultured with tCD19-UpDC^{ENHANCE} (allogeneic and autologous) become highly proliferative compared with the Mock UpDC and CAR T alone conditions. Furthermore, at lower T cell: ENHANCE cell ratio of 1:1 or 1:0.2 co-culture ratios UpDCs^{ENHANCE} boost CAR T cell proliferations significantly better than co-culture with target tumor cells. These data are supporting the robustness of the UpDCs^{ENHANCE} in the fact that the proliferation boosting effect of is observed for both anti-CD19 CART (FIG. 9B) and anti-HER2 CART cells (FIG. 19C).

Thus, this example demonstrates that CAR-antigen expressing UpDC^{ENHANCE} cells potently and specifically enhance CAR T cell proliferation.

### Example 6

Anti-CD19-CAR T cells or Mock T cells were co-cultured with mock U-pDCs, tCD19-U-pDCs^{ENHANCE} cells, or tCD19-K562 tumour cells. After 24 hours of pre-conditioning co-culture, the T cells were immunomagnetically purified by negative selection and repeatedly challenged with CD19-expressing target tumour cells (NALM6) in another cell culture assay, where new NALM6 tumour cells were added every 48-72 hours for a total of three challenges. Tumour cell control by the CAR T cells was analysed 48-72 hours post each tumour cell challenge by flow cytometric counting of the number of live tumour cells (FIG. 10A). The expression of T cell exhaustion markers was analyzed by flow cytometry at 48 h after the third tumour cell challenge (FIG. 10B).

FIG. 10A shows that tCD 19-U-pDC^{ENHANCE} cells can pre-condition anti-CD19 CAR T cells and provide them with a superior ability to restrict tumour expansion after multiple tumour challenges compared with controls. FIG. 10B shows that antiCD19 CAR T cells preconditioned with tCD19-U-pDC^{ENHANCE} cells display a reduced T cell exhaustion phenotype (PD-1+, TIM-3+ cells) compared with control pre-conditioning.

This example demonstrates that CAR T pre-conditioning with CAR-antigen expressing U-pDCs^{ENHANCE} cells results in a CAR T cell population with enhanced ability to kill tumour cells despite multiple challenges and with better protection against exhaustion.

### Example 7

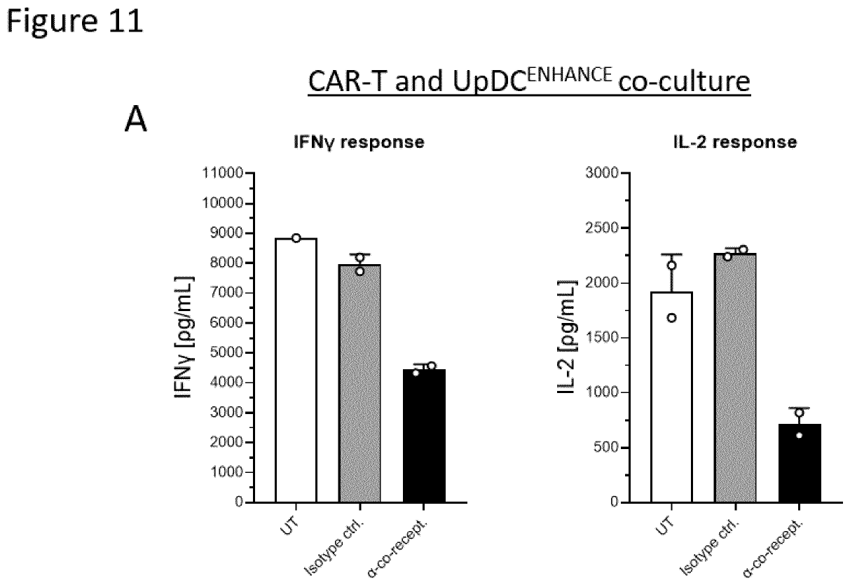

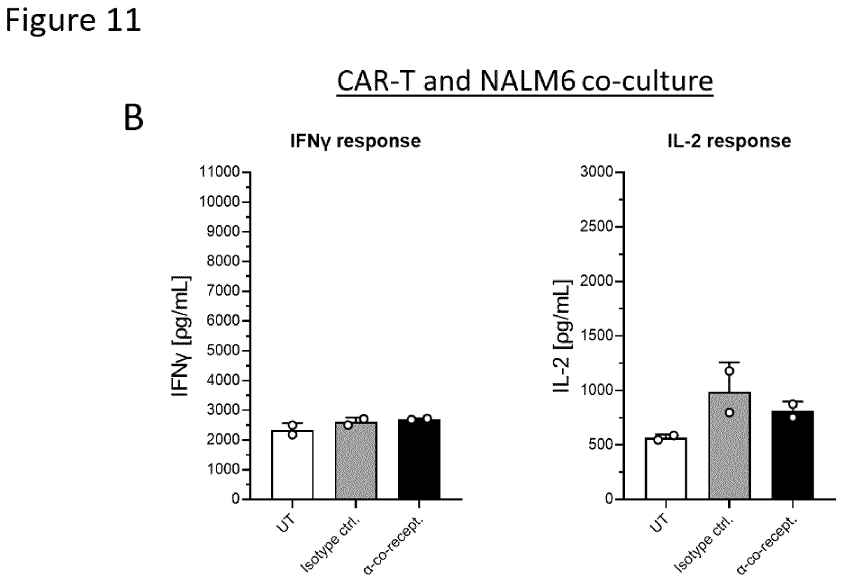

UpDCs^{ENHANCE} facilitates delivery of potent immune activating signals to the CAR T cells through the co-stimulatory receptors naturally expressed on UpDCs (FIG. 3). Using neutralizing antibodies targeting the co-stimulatory receptors on UpDCs the delivery immune activating signals to the CAR T cells can be blocked. The targeted co-stimulatory receptors in this example do not comprise a complete list. FIG. 11A shows that blocking selected co-stimulatory receptors (CD40, CD80, CD86, and ICAM-1) simultaneously greatly decreases UpDCs^{ENHANCE}-induced CAR T activation (based on the secretion of activation associated cytokines: IL-2 and IFNγ) compared with antibody isotype control and untreated conditions. This effect is specific to UpDCs^{ENHANCE}, as specific target tumor cell-induced CAR T activation was not prevented by blocking the co-stimulatory receptors (FIG. 11B).

This example demonstrates that UpDCs^{ENHANCE} facilitates delivery of potent immune activating signals to the CAR T cells through a mode-of-activation involving co-stimulatory receptor interactions between T cells and UpDCs.

### Example 8

Creating a “hot” tumor environment by activating and attracting CAR T cells and endogenous immune cells to the tumor site is essential for tumor regression (FIG. 4). One critical chemokine in the recruitment of immune cells to tumors is CXCL10, which is naturally produced by DCs, macrophages, and pDCs in response to the IFNγ (e.g. from activated T cells).

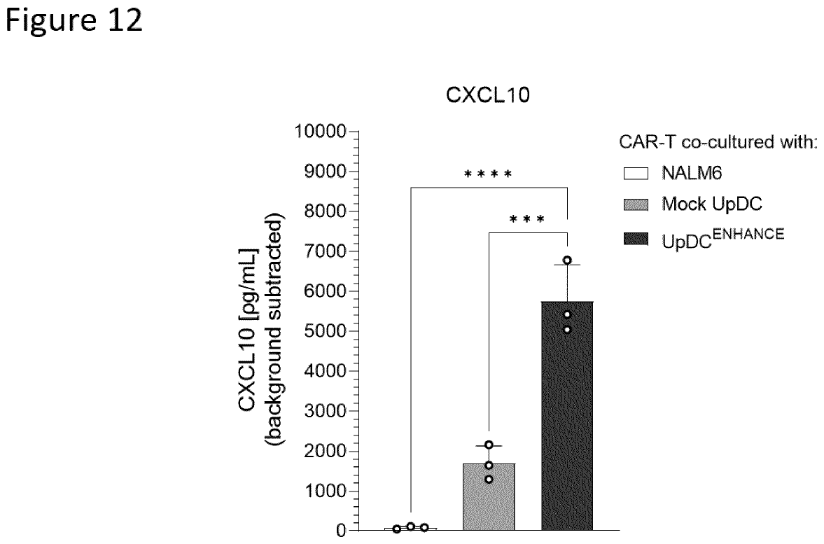

FIG. 12 shows that CAR T cells and UpDCs^{ENHANCE} co-culture greatly increases the secretion of CXCL10 compared with CAR T cells and Mock UpDC or NALM6 co-cultures. This example demonstrates that UpDCs^{ENHANCE} and, CAR T cell interaction facilitates secretion of immune cell recruiting signals.

### Example 9

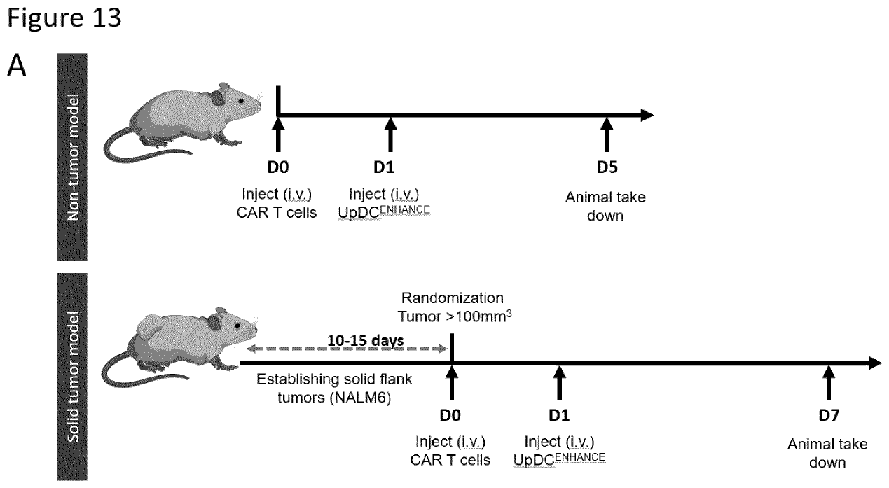

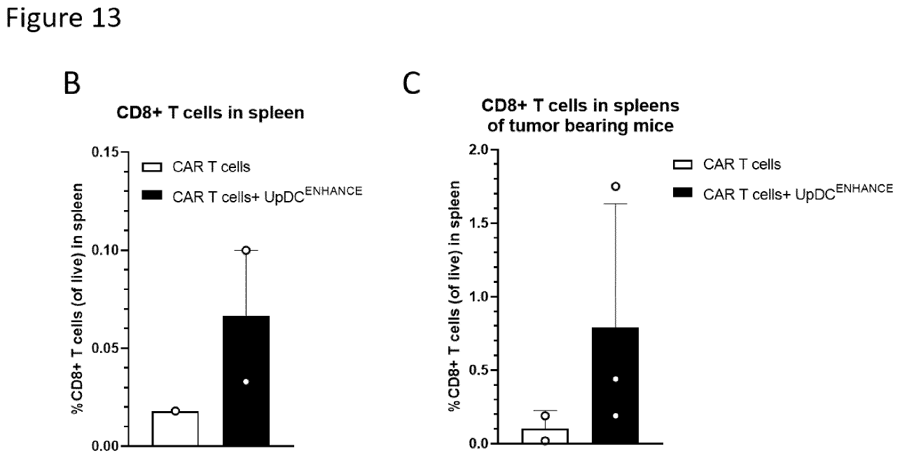

UpDC^{ENHANCE} treatment can expand CAR T cells in vivo. CAR T were intravenously administrated into NXG mice with or without flank tumors with matched tumor antigen (CD19) (FIG. 13A). Twenty-four hours post CAR-T infusion, mice were then intravenously administered with either UpDCs^{ENHANCE} cells or PBS. The percentage of CD8⁺ T cells in the mouse spleens was analyzed by flow cytometry 5 and 7 days after UpDC^{ENHANCE} injection (FIG. 13A). UpDCs^{ENHANCE}-treatment greatly boosted the CAR T cell frequency compared to PBS treatment both non-tumor bearing (FIG. 13B) and tumor-bearing mice (FIG. 13C).

This example demonstrates that UpDCs^{ENHANCE} treatment also enhances CAR T cell proliferation in vivo.

### Example 10

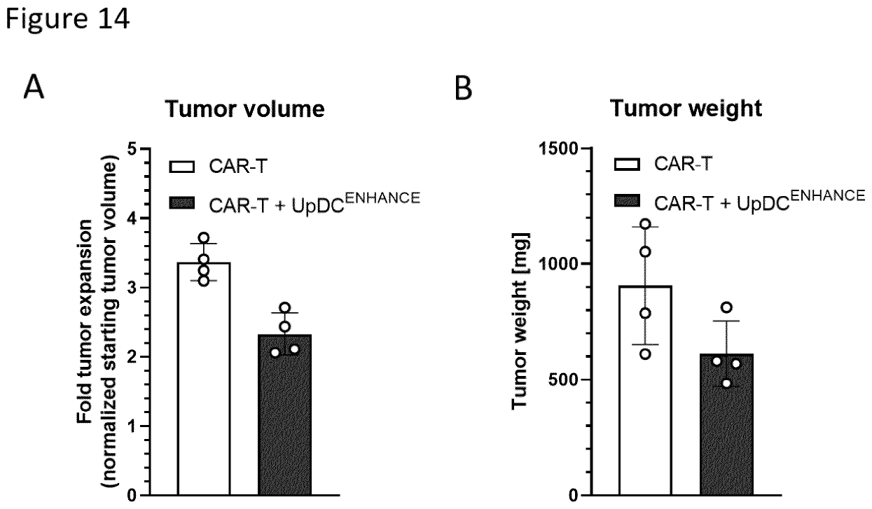

CAR T cells are prone for exhaustion due to the immune-suppressing microenvironment associated with solid tumors (FIG. 3). Mimicking a failing therapy, anti-CD19 CAR T cells were injected (i.v.) at a suboptimal dose into NXG mice with established NALM6 flank tumors. After 24 h the mice were treated (i.v.) with either UpDCs^{ENHANCE} cells or PBS. After seven days, the expansion in tumor volume (FIG. 14A), and the endpoint tumor weight (FIG. 14B) was determined.

FIG. 14 shows that UpDCs^{ENHANCE} treatment decreased tumor expansion both in terms of volume (FIG. 14A) and weight (FIG. 14B). Thus, this example demonstrates that UpDCs ENHANCE treatment improves CAR T cell therapy efficacy in vivo.

1. A method for treating a disease in a subject, comprising administering plasmacytoid dendritic cells (pDCs) in combination with an adoptive cell transfer immunotherapy, wherein the pDCs express at least one antigen that is bound by a receptor expressed by the adoptive cell transfer immunotherapy, or wherein the pDCs express at least one receptor that binds an antigen expressed by the adoptive cell transfer immunotherapy.

2. The method of claim 1, wherein the pDCs are administered simultaneously with the adoptive cell transfer immunotherapy, or wherein the pDCs are administered separately from the adoptive cell transfer immunotherapy, such as wherein the pDCs are administered before the adoptive cell transfer immunotherapy, or wherein the adoptive cell transfer immunotherapy is administered before the pDCs. 3 A method for treating a disease in a subject comprising administering an adoptive cell transfer immunotherapy, wherein the method comprises a step of pre-conditioning the adoptive cell transfer immunotherapy before administration, and wherein the step of pre-conditioning comprises co-culturing the adoptive cell transfer immunotherapy with pDCs expressing at least one antigen that is bound by a receptor expressed by the adoptive cell transfer immunotherapy, or with pDCs expressing at least one receptor that binds an antigen expressed by the adoptive cell transfer immunotherapy.

4. A method of pre-conditioning an adoptive cell transfer immunotherapy comprising co-culturing the adoptive cell transfer immunotherapy with pDCs expressing at least one antigen that is bound by a receptor expressed by the adoptive cell transfer immunotherapy, or with pDCs expressing at least one receptor that binds an antigen expressed by the adoptive cell transfer immunotherapy.

5. The method of claim 3 or claim 4, wherein the ratio of adoptive cell transfer immunotherapy cells to pDCs in the co-culturing is between 2:1 and 1:2, such as between 1.5:1 and 1:1.5, or between 1.2:1 and 1:1.2, or preferably about 1:1, or wherein the ratio of adoptive cell transfer immunotherapy cells to pDCs in the co-culturing is between 1:0.4 and 1:01, such as between 1:0.3 and 1:0.1, or preferably about 1:0.2.

6. The method of any claims 3 to 5, wherein the step of pre-conditioning increases the levels of cytokines produced by the adoptive cell transfer immunotherapy, optionally wherein the cytokines comprise IL-2, TFNα, and/or IFNγ, or wherein the step of pre-conditioning increases the proliferation of the adoptive cell transfer immunotherapy, or wherein the step of pre-conditioning decreases the level of exhaustion, such as measured by expression of exhaustion markers, of the adoptive cell transfer immunotherapy.

7. The method of any one of claims 3-6, further comprising a step of purifying the adoptive cell transfer immunotherapy.

8. The method of any one of claims 4-18, further comprising a step of formulating the adoptive cell transfer immunotherapy with a pharmaceutically acceptable excipient.

9. The method of any one of the preceding claims, wherein the disease is:
(a) a cancer;
(b) an autoimmune disease;
(c) an inflammatory disease; or
(d) an infectious disease.

10. The method of claim 9a, wherein the pDCs express at least one antigen that is bound by a receptor expressed by the adoptive cell transfer immunotherapy and the at least one antigen is a tumour-associated antigen expressed by the cancer to be treated.

11. The method of claim 9b or 9c, wherein the pDCs express at least one receptor that binds an antigen expressed by the adoptive cell transfer immunotherapy and the antigen is recognised by auto-reactive antibodies that cause the autoimmune or inflammatory disease.

12. The method of claim 9b or 9c, wherein the pDCs express at least one antigen that is bound by a receptor expressed by the adoptive cell transfer immunotherapy and the antigen is expressed on the surface of immune cells that cause the autoimmune or inflammatory disease.

13. The method of claim 9(d), wherein the pDCs express at least one antigen that is bound by a receptor expressed by the adoptive cell transfer immunotherapy and the at least one antigen is a pathogen antigen.

14. The method of any of the preceding claims, wherein the pDCs express at least one antigen that is bound by a receptor expressed by the adoptive cell transfer immunotherapy and wherein the at least one antigen is modified to disrupt its signalling activity.

15. The method of any one of the preceding claims, wherein the pDCs express at least one antigen that is bound by a receptor expressed by the adoptive cell transfer immunotherapy and wherein the at least one antigen is truncated to remove one or more intracellular signalling domains or one or more intracellular effector domains.

16. The method of any one of the preceding claims, wherein the pDCs comprise a heterologous nucleic acid encoding the at least one antigen or at least one receptor.

17. The method of any one of the preceding claims, wherein the adoptive cell transfer immunotherapy comprises T-cells, natural killer cells, dendritic cells, macrophages or tumour-infiltrating lymphocytes (TILs).

18. The method of any one of the preceding claims, wherein the adoptive cell transfer immunotherapy comprises cells expressing a chimeric antigen receptor, a T-cell receptor, a synthetic Notch receptor, or a chimeric auto-antibody receptor.

19. The method of any one of the preceding claims, further comprising a step of producing the pDCs, wherein the step of producing the pDCs comprises the steps of:
(a) transfecting or transducing HSPCs with a vector comprising an expression cassette comprising a transgene encoding the at least one antigen operably linked to a promoter; and
(b) differentiating the HSPCs into pDCs, or further comprising a step of producing the pDCs, wherein the step of producing the pDCs comprises the steps of:
(a) differentiating HSPCs into pDCs; and
(b) transfecting or transducing the pDCs with a vector comprising an expression cassette comprising a transgene encoding the at least one antigen operably linked to a promoter.

20. The method of claim 19, further comprising a step of purifying the pDCs.

21. The method of claim 19 or claim 20, further comprising a step of formulating the pDCs with a pharmaceutically acceptable excipient.

22. A method of treating a disease in a subject comprising administering an adoptive cell transfer immunotherapy to a patient that previously received and/or is scheduled to receive administration of pDCs, wherein the pDCs express at least one antigen that is bound by a receptor expressed by the adoptive cell transfer immunotherapy, or wherein the pDCs express at least one receptor that binds an antigen expressed by the adoptive cell transfer immunotherapy.

23. A method of treating a disease in a subject comprising administering pDCs to a patient that previously received and/or is scheduled to receive administration of an adoptive cell transfer immunotherapy, wherein the pDCs express at least one antigen that is bound by a receptor expressed by the adoptive cell transfer immunotherapy, or wherein the pDCs express at least one receptor that binds an antigen expressed by the adoptive cell transfer immunotherapy.
